(19) United States
(12) Patent Application Publication
(10) Pub. No.: US 2014/0046059 A1
(43) Pub. Date: Feb. 13, 2014

(54) PROCESS FOR THE PREPARATION OF MORPHOLINO SULFONYL INDOLE DERIVATIVES

(71) Applicants: Malcolm Mascarenhas, Mumbai (IN); Shashikant Patil, Mumbai (IN); Ravindra Ashok Janrao, Mumbai (IN); Abhijit Roychowdhury, Mumbai (IN)

(72) Inventors: Malcolm Mascarenhas, Mumbai (IN); Shashikant Patil, Mumbai (IN); Ravindra Ashok Janrao, Mumbai (IN); Abhijit Roychowdhury, Mumbai (IN)

(73) Assignee: PIRAMAL ENTERPRISES LIMITED, Mumbai (IN)

(21) Appl. No.: 14/112,743

(22) PCT Filed: Apr. 19, 2012
(86) PCT No.: PCT/IB2012/051958

§ 371 Date: Oct. 18, 2013

Related U.S. Application Data
(60) Provisional application No. 61/477,937, filed on Apr. 21, 2011.

(51) Int. Cl. C07D 413/14

(52) U.S. Cl. CPC C07D 413/14
USPC 544/130

(57) ABSTRACT

The present invention relates to a process for the preparation of the compounds of formula (I) which are morpholino sulphonyl indole derivatives. The compounds of formula (I) are capable of inhibiting, modulating or regulating Insulin-Like-Growth Factor I Receptors or Insulin Receptors. The present invention also relates to the processes for preparation of the pharmaceutically acceptable salts of the compounds of formula (I).

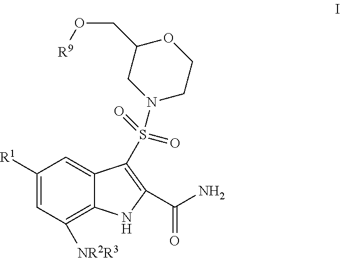

### FIELD OF INVENTION

The present invention relates to a process for the preparation of the compounds of formula (I) which are morpholino sulfonyl indole derivatives. The compounds of formula (I) are capable of inhibiting, modulating or regulating Insulin-Like-Growth Factor I Receptors or Insulin Receptors.

### BACKGROUND OF THE INVENTION

Protein kinases (PKs) are enzymes that catalyze the phosphorylation of hydroxy groups on tyrosine, serine and threonine residues of proteins. The consequences of this seemingly simple activity are staggering; cell growth, differentiation and proliferation; i.e., virtually all aspects of cell life, in one way or another depend on PK activity. Furthermore, abnormal PK activity has been related to a host of disorders, ranging from relatively non life-threatening diseases such as psoriasis to extremely virulent diseases such as glioblastoma (brain cancer). PKs can be broken into two classes, the protein tyrosine kinases (PTKs) and the serine-threonine kinases (STKs).

Certain growth factor receptors exhibiting PK activity are known as receptor tyrosine kinases (RTKs). They comprise a large family of transmembrane receptors with diverse biological activity. At present, at least nineteen (19) distinct subfamilies of RTKs have been identified. One RTK subfamily contains the insulin receptor (IR), insulin-like growth factor I receptor (IGF-1R) and insulin receptor related receptor (IRR). IR and IGF-1R interact with insulin to activate a hetero-tetramer composed of two entirely extracellular glycosylated a subunits and two β subunits which cross the cell membrane and which contain the tyrosine kinase domain. The Insulin-like Growth Factor-1 Receptor (IGF-1R), and its ligands, IGF-1 and IGF-2, are abnormally expressed in numerous tumors, including, but not limited to, breast, prostate, thyroid, lung, hepatoma, colon, brain, neuroendocrine, and others.

Numerous IGF-1R small molecule inhibitors have been found to inhibit cancer growth in vitro, in vivo and in clinical trials. For example, BMS-754807 effectively inhibits the growth of a broad range of human tumor types in vitro, including mesenchymal (Ewing's, rhabdomyosarcoma, neuroblastoma, and lipo sarcoma), epothelial (breast, lung, pancreatic, colon, gastric), and hematopoietic (multiple myeloma and leukemia) tumor cell lines. Carboni et al., Mol Cancer Ther 2009; 8(12).

Various morpholino sulfonyl indole derivatives that are capable of inhibiting, modulating or regulating Insulin-Like-Growth Factor I Receptors or Insulin Receptors have been disclosed in the applicant's co-pending PCT patent application. These compounds find application in the treatment of diseases or disorders mediated by Insulin-Like-Growth Factor I Receptors or Insulin Receptors such as cancer. The current PCT patent application provides processes for the preparation of said compounds represented herein as the compounds of formula (I).

### SUMMARY OF THE INVENTION

In one aspect, the present invention relates to a process for the preparation of a compound of formula I, a stereoisomer or pharmaceutically accepatable salt thereof. In another aspect, the present invention relates to a process for the preparation of a compound of formula I, particularly a pharmaceutically acceptable salt namely methane sulfonate of (S)-ethyl 4-(2-carbamoyl-5-chloro-3-(2-(phenoxymethyl) morpholinosulfonyl)-1H-indol-7-ylamino) piperidine-1-carboxylate.

### DETAILED DESCRIPTION OF THE INVENTION

The present invention provides a process for the preparation of a compound of formula I. The compounds of this invention are useful in the inhibition of IGF-1R or IR.

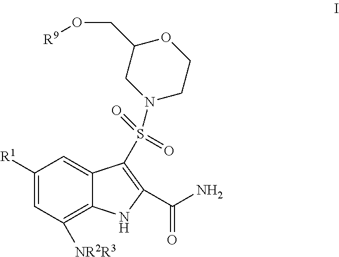

wherein:
R^{a} is independently selected from the group consisting of H and C₁-C₆ alkyl, said alkyl is optionally substituted with one to three substituents selected from R⁷;
R¹ is selected from the group consisting of:
H, Halogen, NO₂, CN, (CR^{a}₂)ₙOR⁵, (CR^{a}₂)ₙN(R⁵)₂, C(O)R⁵, C(O)OR₅, (CR^{a}₂)ₙR⁵, S(O)ₘR₅, S(O)ₘN(R⁵)₂, SR⁵, OS(O)ₘR⁵, N(R⁵)C(O)R⁵, N(R⁵)S(O)ₘR⁵, and (CR^{a}₂)ₙC(O)N(R⁵)₂;
R² is H or C₁-C₆ alkyl;
R³ is —C(Z)—X—C(O)—Y, —X—Y, —C(Z)—NR⁸R¹¹ or heterocyclyl, wherein said heterocyclyl is optionally substituted with one to three substituents selected from the group consisting of C₁-C₆ alkyl, NR⁸C(O)R¹⁰, C(O)NR⁸R¹⁰ and C(O)OR¹²;
R⁵ is independently selected from the group consisting of:
H, C₆-C₁₀aryl, 5-10 membered heterocyclyl, 5-10 membered heterocyclenyl, 5-10 membered heteroaryl, C₁-C₆ alkyl, and C₃-C₈ cycloalkyl,
said aryl, heterocyclyl, heterocyclenyl, heteroaryl, alkyl and cycloalkyl is optionally substituted with one to three substituents selected from R⁷;
R⁷ is independently selected from the group consisting of: C₁-C₆ alkyl, Halogen, C₁-C₆ alkoxy, C₁-C₆ haloalkyl, CN, NH₂, and NO₂;
R⁸ is independently H or C₁-C₆ alkyl;
R⁹ is selected from the group consisting of C₆-C₁₀aryl, 5-10 membered heterocyclyl, 5-10 membered heterocyclenyl and 5-10 membered heteroaryl, said aryl, heterocyclyl, heterocyclenyl, heteroaryl, is optionally substituted with one to three substituents selected from R⁷;
R¹⁰ is independently selected from the group consisting of C₃-C₈cycloalkyl, C₁-C₆alkyl, and C₃-C₈cycloalkylC₁-C₃alkyl,
R¹¹ is selected from the group consisting of H, C₁-C₆ alkyl, C₆-C₁₀aryl, 5-10 membered heterocyclyl, 5-10 membered heterocyclenyl, and C₃-C₈cycloalkyl, optionally substituted with one to three substituents selected from R⁷;
R¹² is H or C₁-C₆ alkyl;
X is C₂-C₆ alkylene or C₃-C₈cycloalkylene;
Y is selected from the group consisting of H, OR¹², CN, heterocyclyl, NR⁸R¹⁰, wherein said heterocyclyl is optionally substituted with one to three substituents selected from the group consisting of C(O)NR⁸R¹⁰, NR⁸C(O)R¹⁰, C₁-C₆ alkyl and C(O)OR¹²;

### Z is NH, O or S;

m is 1 or 2; and
n is independently 0, 1, 2, 3, 4, 5 or 6;
or a pharmaceutically acceptable salt thereof.

In another embodiment, the invention provides a process for the preparation of a compound of formula IA, wherein

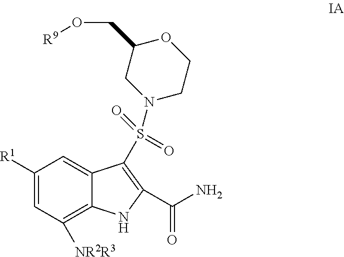

R¹ is halo;

### R² is H;

R³ is —C(O)—X—C(O)—Y, —X—Y, —C(S)—NR¹¹R⁸, or heterocyclyl selected from the group consisting of tetrahydro-pyranyl, piperidinyl and pyrrolidinyl, and wherein the heterocyclyl is optionally substituted with halo, C(O)NR⁸R¹⁰, C₁-C₆ alkyl, or C(O)OR¹²;

### R⁸ is H;

R⁹ is phenyl or pyridyl optionally substituted with one to three substituents selected from R⁷;
R¹⁰ is independently selected from the group consisting of C₃-C₈cycloalkyl, C₁-C₆alkyl, and C₃-C₈cycloalkylC₁-C₃ alkyl;
R¹¹ is phenyl optionally substituted with one to three substituents selected from R⁷;
R¹² is C₁-C₃ alkyl;
X is C₂-C₆ alkylene or C₃-C₈cycloalkylene; and
Y is selected from the group consisting of H, OR¹², CN, morpholinyl, and NH₂, wherein said morpholinyl is optionally substituted with C(O)NR⁸R¹⁰, C₁-C₆ alkyl, or C(O)OR¹².

In a further embodiment, the invention provides a process for the preparation of compound of Formula II,

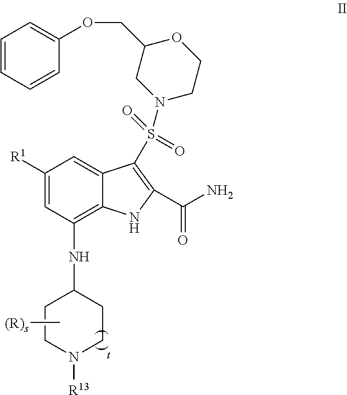

wherein R¹ is halo;
R¹³ is selected from the group consisting of H, C(O)NR⁸R¹⁰, C₁-C₆ alkyl, and C(O)OR¹²;
R⁸ is H or C₁-C₃ alkyl;
R¹⁰ is selected from the group consisting of C₃-C₈cycloalkyl, C₁-C₆alkyl, and C₃-C₈cycloalkylC₁-C₃alkyl,
R¹² is H or C₁-C₃ alkyl;
R is halo;
s is 0, 1, 2, 3, or 4; and
t is 0 or 1.

In a further embodiment, the invention provides a process for the preparation of compound of Formula IIA:

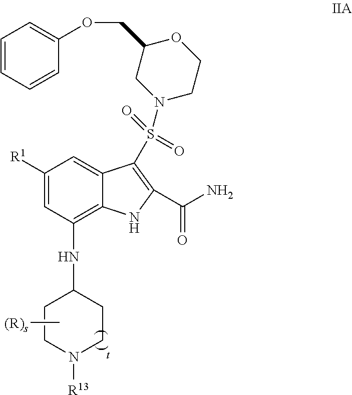

wherein R¹ is halo;

### R¹³ is C(O)OR¹²;

R¹² is H or C₁-C₃ alkyl;
R is halo;
s is 0, 1, 2, 3, or 4; and
t is 0 or 1.

In an embodiment, the present invention provides a process for the preparation of a compound selected from:
(S)-4-(2-Carbamoyl-5-chloro-3-(2-(phenoxymethyl)morpholinosulfonyl)-1H-indol-7-ylamino)-4-oxobutanoic acid;
(S)-5-(2-Carbamoyl-5-chloro-3-(2-(phenoxymethyl)morpholinosulfonyl)-1H-indol-7-ylamino)-3,3-dimethyl-5-oxopentanoic acid;
(S)-4-(2-Carbamoyl-5-chloro-3-(2-(phenoxymethyl)morpholinosulfonyl)-1H-indol-7-ylamino)-2,2-dimethyl-4-oxobutanoic acid;
(S)-5-(2-Carbamoyl-5-chloro-3-(2-(phenoxymethyl)morpholinosulfonyl)-1H-indol-7-ylamino)-5-oxopentanoic acid;
2-(2-Carbamoyl-5-chloro-3-((S)-2-(phenoxymethyl)morpholinosulfonyl)-1H-indol-7-ylcarbamoyl)cyclopropanecarboxylic acid;
(S)-5-Chloro-7-(5-morpholino-5-oxopentanamido)-3-(2-(phenoxymethyl)morpholino sulfonyl)-1H-indole-2-carboxamide;
(S)-5-Chloro-7-(2-cyanoacetamido)-3-(2-(phenoxymethyl)morpholinosulfonyl)-1H-indole-2-carboxamide;
(S)-Ethyl 5-(2-carbamoyl-5-chloro-3-(2-(phenoxymethyl)morpholinosulfonyl)-1H-indol-7-ylamino)-5-oxopentanoate;
(S)-3-(2-Carbamoyl-5-chloro-3-(2-(phenoxymethyl)morpholinosulfonyl)-1H-indol-7-ylamino)propanoic acid;
(S)-7-(3-Amino-3-oxopropylamino)-5-chloro-3-(2-(phenoxymethyl)morpholinosulfonyl)-1H-indole-2-carboxamide;
(S)-Ethyl 4-(2-carbamoyl-5-chloro-3-(2-(phenoxymethyl)morpholinosulfonyl)-1H-indol-7-ylamino)butanoate;
(S)-5-Chloro-7-(2-cyanoethylamino)-3-(2-(phenoxymethyl)morpholinosulfonyl)-1H-indole-2-carboxamide;
(S)-5-Chloro-3-(2-(phenoxymethyl)morpholinosulfonyl)-7-(tetrahydro-2H-pyran-4-ylamino)-1H-indole-2-carboxamide;
(S)-5-Chloro-7-(cyclohexylamino)-3-(2-(phenoxymethyl)morpholinosulfonyl)-1H-indole-2-carboxamide;
(S)-5-Chloro-7-(cyclohexylmethylamino)-3-(2-(phenoxymethyl)morpholinosulfonyl)-1H-indole-2-carboxamide;
(S)-Methyl 4-((2-carbamoyl-5-chloro-3-(2-(phenoxymethyl)morpholinosulfonyl)-1H-indol-7-ylamino)methyl)benzoate;
(S)-5-Chloro-7-(cyclopentylamino)-3-(2-(phenoxymethyl)morpholinosulfonyl)-1H-indole-2-carboxamide;
(S)-7-((1-Aminocyclopentyl)methylamino)-5-chloro-3-(2-(phenoxymethyl) morpholino sulfonyl)-1H-indole-2-carboxamide;
(S)-4-((2-Carbamoyl-5-chloro-3-(2-(phenoxymethyl)morpholinosulfonyl)-1H-indol-7-ylamino)methyl)benzoic acid;
(S)-7-(1-(tert-Butylcarbamoyl)piperidin-4-ylamino)-5-chloro-3-(2-(phenoxymethyl)morpholinosulfonyl)-1H-indole-2-carboxamide;
(S)-5-Chloro-7-(1-(cyclohexylcarbamoyl)piperidin-4-ylamino)-3-(2-(phenoxymethyl)morpholinosulfonyl)-1H-indole-2-carboxamide;
(S)-5-Chloro-7-(1-(cyclohexylmethylcarbamoyl)piperidin-4-ylamino)-3-(2-(phenoxymethyl)morpholinosulfonyl)-1H-indole-2-carboxamide;
(S)-5-Chloro-7-(4-fluorobenzylamino)-3-(2-(phenoxymethyl)morpholinosulfonyl)-1H-indole-2-carboxamide;
(S)-5-chloro-7-(1-isobutylpiperidin-4-ylamino)-3-(2-(phenoxymethyl)morpholinosulfonyl)-1H-indole-2-carboxamide;
5-Chloro-3-((S)-2-(phenoxymethyl)morpholinosulfonyl)-7-(pyrrolidin-3-ylamino)-1H-indole-2-carboxamide;
(S)-Ethyl 4-(2-carbamoyl-5-fluoro-3-(2-(phenoxymethyl)morpholinosulfonyl)-1H-indol-7-ylamino)piperidine-1-carboxylate;
(S)-Ethyl 4-(2-carbamoyl-5-chloro-3-(2-(phenoxymethyl)morpholinosulfonyl)-1H-indol-7-ylamino)piperidine-1-carboxylate;
(S)-5-Chloro-3-(2-(phenoxymethyl)morpholinosulfonyl)-7-(3-phenylthioureido)-1H-indole-2-carboxamide; and
(S)-5-Chloro-3-(2-(phenoxymethyl)morpholinosulfonyl)-7-(piperidin-4-ylamino)-1H-indole-2-carboxamide;
   or a pharmaceutically acceptable salt thereof.

### Chemical Definitions:

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on.

When used in the phrases “alkylaryl”, “alkylcycloalkyl” and “alkylheterocyclyl” the term “alkyl” refers to the alkyl portion of the moiety and does not describe the number of atoms in the heterocyclyl portion of the moiety. In an embodiment, if the number of carbon atoms is not specified, the “alkyl” of “alkylaryl”, “alkylcycloalkyl” and “alkylheterocyclyl” refers to C₁-C₁₂ alkyl and in a further embodiment, refers to C₁-C₆ alkyl.

The term “cycloalkyl” means a monocyclic saturated or unsaturated aliphatic hydrocarbon group having the specified number of carbon atoms. The cycloalkyl is optionally bridged (i.e., forming a bicyclic moiety), for example with a methylene, ethylene or propylene bridge. The cycloalkyl may be fused with an aryl group such as phenyl, and it is understood that the cycloalkyl substituent is attached via the cycloalkyl group. For example, “cycloalkyl” includes cyclopropyl, methyl-cyclopropyl, 2,2-dimethyl-cyclobutyl, 2-ethyl-cyclopentyl, cyclohexyl, cyclopentenyl, cyclobutenyl and so on.

In an embodiment, if the number of carbon atoms is not specified, “alkyl” refers to C₁-C₁₂ alkyl and in a further embodiment, “alkyl” refers to C₁-C₆ alkyl. In an embodiment, if the number of carbon atoms is not specified, “cycloalkyl” refers to C₃-C₁₀ cycloalkyl and in a further embodiment, “cycloalkyl” refers to C₃-C₇ cycloalkyl. In an embodiment, examples of “alkyl” include methyl, ethyl, n-propyl, i-propyl, n-butyl, t-butyl and i-butyl.

The term “alkylene” means a hydrocarbon diradical group having the specified number of carbon atoms. For example, “alkylene” includes —CH₂—, —CH₂CH₂— and the like.

In an embodiment, if the number of carbon atoms is not specified, “alkylene” refers to C₁-C₁₂ alkylene and in a further embodiment, “alkylene” refers to C₁-C₆ alkylene.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

“Alkenylene” means a diradical group of an alkenyl group that is defined above. For example, “alkenylene” includes —CH₂—CH₂—CH═CH—CH₂, —CH═CH—CH₂ and the like.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

“Aryl” is intended to mean any stable monocyclic, bicyclic or tricyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

In one embodiment, “aryl” is an aromatic ring of 6 to 14 carbons atoms, and includes a carbocyclic aromatic group fused with a 5- or 6-membered cycloalkyl group such as indan. Examples of carbocyclic aromatic groups include, but are not limited to, phenyl, naphthyl, e.g. 1-naphthyl and 2-naphthyl; anthracenyl, e.g. 1-anthracenyl, 2-anthracenyl; phenanthrenyl; fluorenonyl, e.g. 9-fluorenonyl, indanyl and the like.

The term heteroaryl, as used herein, represents a stable monocyclic, bicyclic or tricyclic ring of up to 7 atoms in each ring, wherein at least one ring is aromatic and contains carbon and from 1 to 4 heteroatoms selected from the group consisting of O, N and S. In another embodiment, the term heteroaryl refers to a monocyclic, bicyclic or tricyclic aromatic ring of 5- to 14-ring atoms of carbon and from one to four heteroatoms selected from O, N, or S. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

Heteroaryl groups within the scope of this definition include but are not limited to acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. Additional examples of heteroaryl include, but are not limited to pyridyl, e.g., 2-pyridyl (also referred to as α-pyridyl), 3-pyridyl (also referred to as β-pyridyl) and 4-pyridyl (also referred to as (γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like.

In an embodiment, “heteroaryl” may also include a “fused polycyclic aromatic”, which is a heteroaryl fused with one or more other heteroaryl or nonaromatic heterocyclic ring. Examples include, quinolinyl and isoquinolinyl, e.g. 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl, e.g. 2-benzofuranyl and 3-benzofuranyl; dibenzofuranyl, e.g. 2,3-dihydrobenzofuranyl; dibenzothiophenyl; benzothienyl, e.g. 2-benzothienyl and 3-benzothienyl; indolyl, e.g. 2-indolyl and 3-indolyl; benzothiazolyl, e.g., 2-benzothiazolyl; benzooxazolyl, e.g., 2-benzooxazolyl; benzimidazolyl, e.g. 2-benzoimidazolyl; isoindolyl, e.g. 1-isoindolyl and 3-isoindolyl; benzotriazolyl; purinyl; thianaphthenyl, pyrazinyland the like.

“Heterocyclyl” means a non-aromatic saturated monocyclic, bicyclic, tricyclic or spirocyclic ring system comprising up to 7 atoms in each ring. Preferably, the heterocyclyl contains 3 to 14, or 5 to 10 ring atoms, in which one or more of the atoms in the ring system is an element other than carbon, for example, nitrogen, oxygen, phosphor or sulfur, alone or in combination. There are no adjacent oxygen and/or sulfur atoms present in the ring system. Preferred heterocyclyls contain about 5 to about 6 ring atoms. The heterocycle may be fused with an aromatic aryl group such as phenyl or heterocyclenyl. The prefix aza, oxa or thia before the heterocyclyl root name means that at least a nitrogen, oxygen or sulfur atom, respectively, is present as a ring atom. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom. An example of such a moiety is pyrrolidone;

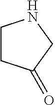

In describing the heteroatoms contained in a specified heterocyclyl group, the expression, “having one to x heteroatoms selected from the group of N, O, P and S” (wherein x is a specified integer), for example, means that each heteroatom in the specified heterocyclyl is independently selected from the specified selection of heteroatoms. Attachment of a heterocyclyl substituent can occur via a carbon atom or via a heteroatom.

“Heterocyclenyl” means a non-aromatic monocyclic, bicyclic, tricyclic or spirocyclic ring system comprising up to 7 atoms in each ring. Preferably, the heterocyclenyl contains 3 to 14, or 5 to 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen, phosphor or sulfur atom respectively is present as a ring atom. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes heterocyclenyl rings as described above wherein ═O replaces two available hydrogens on the same ring carbon atom. An example of such a moiety is pyrrolidinone;

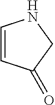

In describing the heteroatoms contained in a specified heterocyclenyl group, the expression, “having one to x heteroatoms selected from the group of N, O, P and S” (wherein x is a specified integer), for example, means that each heteroatom in the specified heterocyclenyl is independently selected from the specified selection of heteroatoms.

It should also be noted that tautomeric forms such as, for example, the moieties;

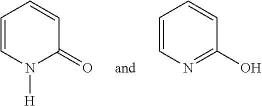 [SMILES: O=c1cccc[nH]1 ; "and" ; Oc1ccccn1]

are considered equivalent in certain embodiments of this invention.

An “alkylaryl group” is an alkyl group substituted with an aryl group, for example, a phenyl group. Suitable aryl groups are described herein and suitable alkyl groups are described herein. The bond to the parent moiety is through the aryl group.

An “alkylheteroaryl group” is an alkyl group substituted with a heteroaryl group. Suitable heteroaryl groups are described herein and suitable alkyl groups are described herein. The bond to the parent moiety is through the heteroaryl group.

An “alkylheterocyclyl group” is an alkyl group substituted with a heterocyclyl group. Suitable heterocyclyl groups are described herein and suitable alkyl groups are described herein. The bond to the parent moiety is through the heterocyclyl group.

An “alkylheterocyclenyl group” is an alkyl group substituted with a heterocyclenyl group. Suitable heterocyclenyl groups are described herein and suitable alkyl groups are described herein. The bond to the parent moiety is through the heterocyclenyl group.

An “alkylcycloalkyl group” is an alkyl group substituted with a cycloalkyl group. Suitable cycloalkyl groups are described herein and suitable alkyl groups are described herein. The bond to the parent moiety is through the cycloalkyl group.

An “arylalkyl group” is an aryl group substituted with an alkyl group, for example, a phenyl group. Suitable aryl groups are described herein and suitable alkyl groups are described herein. The bond to the parent moiety is through the alkyl group.

A “heteroarylalkyl group” is a heteroaryl group substituted with an alkyl group. Suitable heteroaryl groups are described herein and suitable alkyl groups are described herein. The bond to the parent moiety is through the alkyl group.

A “heterocyclylalkyl group” is a heterocyclyl group substituted with an alkyl group. Suitable heterocyclyl groups are described herein and suitable alkyl groups are described herein. The bond to the parent moiety is through the alkyl group.

A “heterocyclenylalkyl group” is a heterocyclenyl group substituted with an alkyl group. Suitable heterocyclenyl groups are described herein and suitable alkyl groups are described herein. The bond to the parent moiety is through the alkyl group.

A “cycloalkylalkyl group” is a cycloalkyl group substituted with an alkyl group. Suitable cycloalkyl groups are described herein and suitable alkyl groups are described herein. The bond to the parent moiety is through the alkyl group.

An “aryloxy group” is an aryl group that is attached to a compound via an oxygen (e.g., phenoxy).

An “alkoxy group” (alkyloxy), as used herein, is a straight chain or branched C₁-C₁₂ or cyclic C₃-C₁₂ alkyl group that is connected to a compound via an oxygen atom. Examples of alkoxy groups include but are not limited to methoxy, ethoxy and propoxy.

An “arylalkoxy group” (arylalkyloxy) is an arylalkyl group that is attached to a compound via an oxygen on the alkyl portion of the arylalkyl (e.g., phenylmethoxy).

An “arylamino group” as used herein, is an aryl group that is attached to a compound via a nitrogen.

An “alkylamino group” as used herein, is an alkyl group that is attached to a compound via a nitrogen.

As used herein, an “arylalkylamino group” is an arylalkyl group that is attached to a compound via a nitrogen on the alkyl portion of the arylalkyl.

An “alkylsulfonyl group” as used herein, is an alkyl group that is attached to a compound via the sulfur of a sulfonyl group.

When a moiety is referred to as “unsubstituted” or not referred to as “substituted” or “optionally substituted”, it means that the moiety does not have any substituents. When a moiety is referred to as substituted, it denotes that any portion of the moiety that is known to one skilled in the art as being available for substitution can be substituted. The phrase “optionally substituted with one or more substituents” means, in one embodiment, one substituent, two substituents, three substituents, four substituents or five substituents. For example, the substitutable group can be a hydrogen atom that is replaced with a group other than hydrogen (i.e., a substituent group). Multiple substituent groups can be present. When multiple substituents are present, the substituents can be the same or different and substitution can be at any of the substitutable sites. Such means for substitution are well known in the art. For purposes of exemplification, which should not be construed as limiting the scope of this invention, some examples of groups that are substituents are: alkyl, alkenyl or alkynyl groups (which can also be substituted, with one or more substituents), alkoxy groups (which can be substituted), a halogen or halo group (F, Cl, Br, I), hydroxy, nitro, oxo, —CN, —COH, —COOH, amino, azido, N-alkylamino or N,N-dialkylamino (in which the alkyl groups can also be substituted), N-arylamino or N,N-diarylamino (in which the aryl groups can also be substituted), esters (—C(O)—OR, where R can be a group such as alkyl, aryl, etc., which can be substituted), ureas (—NHC(O)—NHR, where R can be a group such as alkyl, aryl, etc., which can be substituted), carbamates (—NHC(O)—OR, where R can be a group such as alkyl, aryl, etc., which can be substituted), sulfonamides (—NHS(O)₂R, where R can be a group such as alkyl, aryl, etc., which can be substituted), alkylsulfonyl (which can be substituted), aryl (which can be substituted), cycloalkyl (which can be substituted) alkylaryl (which can be substituted), alkylheterocyclyl (which can be substituted), alkylcycloalkyl (which can be substituted), and aryloxy.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

The present invention also encompasses within its scope a process for the preparation of the pharmaceutically acceptable salt of the compounds of formula (I). It is well known that for use in medicine, the compounds of Formula I may be required to be provided as their pharmaceutically acceptable salts. When the compound of formula (I) is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of formula (I) is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. In one embodiment, the acids are citric, hydrobromic, hydrochloric, maleic, phosphoric, sulfuric or tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of formula (I) are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

Abbreviations, which may be used in the description of the chemistry and in the Examples that follow, include
Ac₂O Acetic anhydride;
AcOH Acetic acid;
Ar Aryl;
AlCl₃ Aluminium chloride;
BF₃ Boron trifluoride;
CDCl₃ Deuterated chloroform;
Bn Benzyl;
BOC/Boc tert-Butoxycarbonyl;
DCM Dichloromethane;
DMAP 4-Dimethylaminopyridine;
DMF N,N-Dimethylformamide;
DMSO Dimethyl sulfoxide;
DMSO-d₆ Deuterated dimethyl sulfoxide;
D₂O Deuterated water;
EDTA Ethylenediaminetetraacetic acid;
Et₃N Triethylamine;
EtOAc Ethyl acetate;
EtOH Ethanol;
Fe Iron;
HCl Hydrochloric acid;
HPLC High-performance liquid chromatography;
KOH Potassium hydroxide;
Me Methyl;
MeOH Methanol;
Ms Methanesulfonyl;
MS Mass Spectroscopy;
MsCl Methanesulfonyl chloride;
n-Bu n-butyl;
NH₃ Ammonia
NaOH Sodium hydroxide;
NaOEt Sodium ethoxide;
Na₂SO₄ Sodium sulfate;
NaNO₂ Sodium nitrite;
NH₄Cl Ammonium chloride;
NMR Nuclear Magnetic Resonance;
Ph Phenyl;
Py or pyr Pyridine;
Pd/C Palladium over activated charcoal or Palladium-carbon;
P₂O₅ Phosphorous pentoxide;
SnCl₂ Stannous chloride;
RT Room Temperature;
t-Bu tert-Butyl;
TFA Trifluoroacetic acid;
THF Tetrahydrofuran;
Zn Zinc;
ZnCl₂ Zinc chloride;

The process for the preparation of the compounds of formula (I) according to the present invention employs reactions as shown in the following schemes, in addition to other standard manipulations that are known in the literature or exemplified in the experimental procedures. These schemes, therefore, are not limited by the compounds listed nor by any particular substituents employed for illustrative purposes. Substituent numbering, as shown in the schemes, does not necessarily correlate to that used in the claims

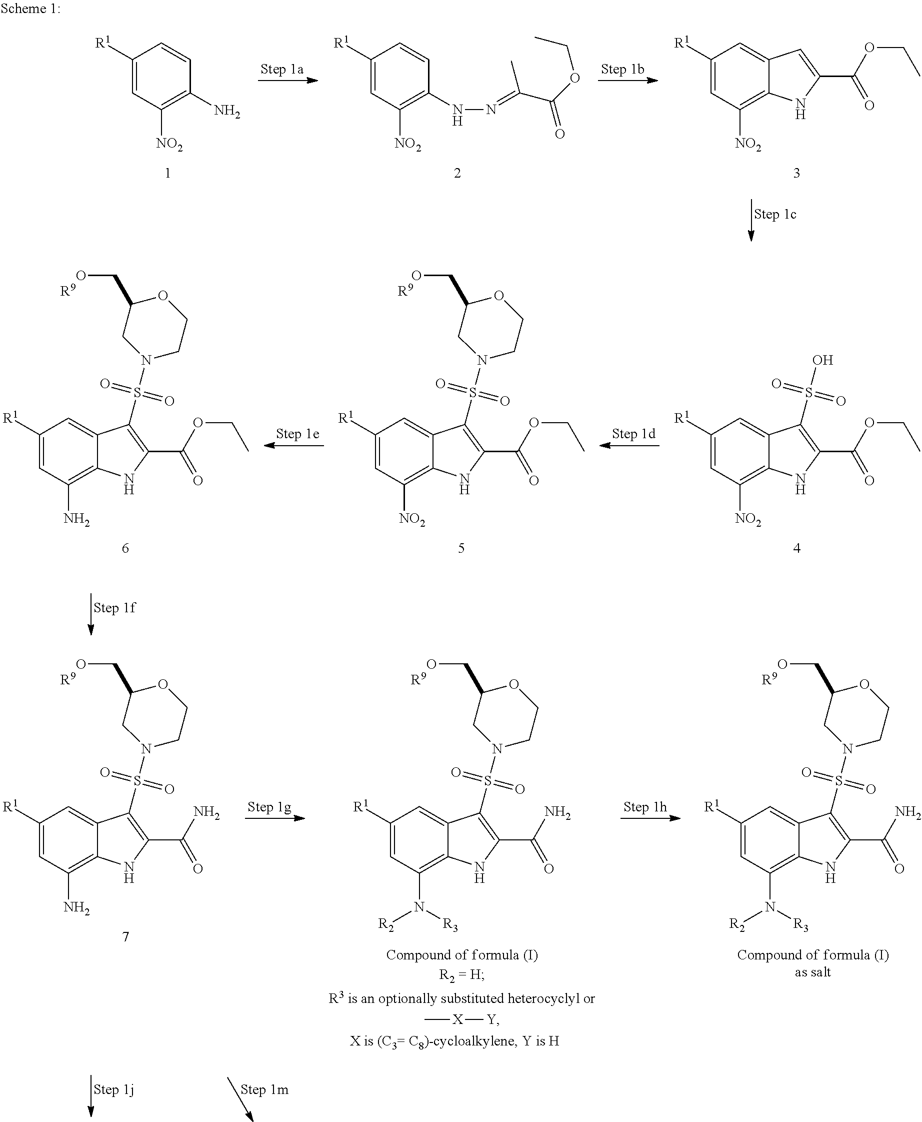

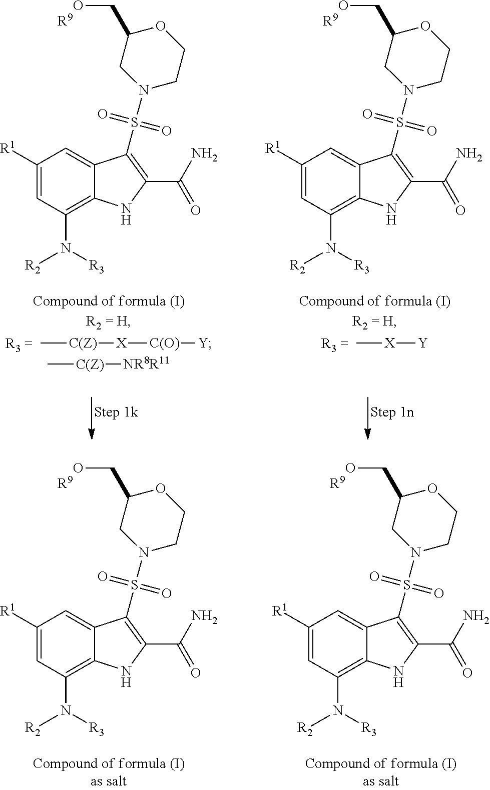

Scheme 1 describes the detailed process for the preparation of the compound of formula 1, the steps comprising:

Step 1a: Diazotising the compound of formula 1 (which is commercially available or may be prepared by methods, well-known in the art);

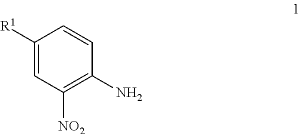

wherein R¹ is as defined in formula I, by reacting it with sodium nitrite (NaNO₂) and HCl at a temperature range of −10 to 5° C., followed by a dropwise addition of the diazotized mixture to an alkaline solution of the reagent, ethyl 2-methyl-3-oxobutanoate in a base selected from potassium hydroxide (KOH) or sodium hydroxide (NaOH) in a solvent such as methanol or ethanol at a temperature range of −20° C. to −15° C. to afford the compound of formula 2;

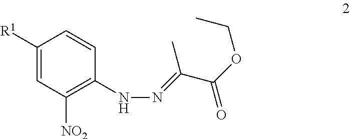

wherein R¹ is as defined in formula I.
Step 1b: Cyclising the compound of formula 2 by reaction with a Lewis acid such as ZnCl₂, AlCl₃, BF₃, P₂O₅ or polyphosphoric acid at a temperature range of 80-120° C. for 5-12 h to afford the compound of formula 3;

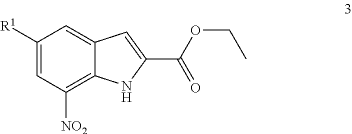

wherein R¹ is as defined in formula I.
Step 1c: Sulphonating the compound of formula 3 by reaction with sulphuric acid and acetic anhydride at a temperature range of 0-30° C. for 10-20 h to afford the compound of formula 4;

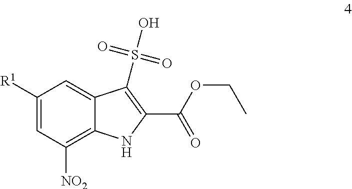

wherein R¹ is as defined in formula I.
Step 1d: Reacting the compound of formula 4 with oxalyl chloride or thionyl chloride in the presence of an organic base selected from triethylamine or pyridine in a solvent selected from DMF, methylene dichloride or a mixture thereof at a temperature range of 25-50° C. for 1-6 h to prepare the corresponding sulphonyl chloride of the compound of formula 4, which is further reacted with the intermediate of formula E;

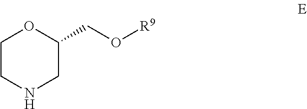

wherein R⁹ is as defined in formula I; at room temperature in presence of an organic base selected from pyridine or triethylamine in a solvent selected from dichloromethane or chloroform at room temperature (25-30° C.) for 2-12 h to afford the compound of formula 5;

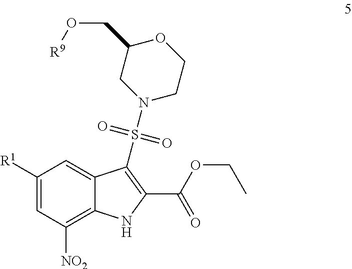

wherein R¹ and R⁹ are as defined in formula I.
Step 1e: Reducing the compound of formula 5 by reaction with a reducing agent selected from Fe and NH₄Cl, Zn and HCl or SnCl₂ for 2-8 h in a suitable solvent selected from methanol, ethanol, THF, water or a mixture thereof, to afford the compound of formula 6;

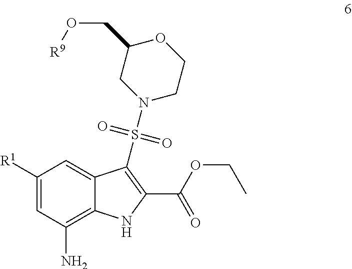

wherein R¹ and R⁹ are as defined in formula I.
Step 1f: Reacting the compound of formula 6 with isopropyl alcohol and ammonia at a temperature range of 80 to 120° C. in a sealed tube for 10-18 h or in a microwave for 10-15 min to afford the compound of formula 7;

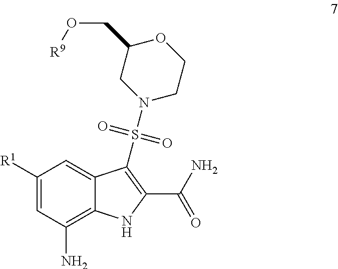

wherein R¹ and R⁹ are as defined in formula I.
Step 1g: Reacting the compound of formula 7 with the reagent of formula F;

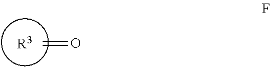

wherein R³ is an optionally substituted heterocyclyl or —X—Y wherein X is (C₃-C₈)-cycloalkylene and Y is H, as defined in Formula I; in presence of trifluoroacetic acid in a suitable base such as sodium triacetoxy borohydride and optionally, Hunig's base; in a suitable solvent selected from dichloromethane or ethyl acetate at room temperature for 0.5-2 h to afford the compound of formula I;

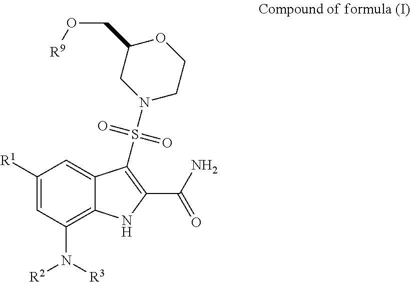

wherein R¹ and R⁹ are as defined in formula I; R² is H and R³ is an optionally substituted heterocyclyl or —X—Y wherein X is (C₃-C₈)-cycloalkylene and Y is H.
Step 1h: Reaction of the compound of formula I obtained in Step 1 g with an acid to afford corresponding pharmaceutically acceptable salt of the compound of formula I of Step 1g.
Step 1j: Reaction of the compound of formula 7 with the compound of formula (R³)₂O, R³OH or R¹¹NC(Z) in a suitable solvent selected from toluene, dioxane or THF at a temperature range of 70° C. to 100° C. for about 1-4 h to afford the compound of formula I, wherein R³ is —C(Z)XC(O)Y or —C(Z)NR⁸R¹¹ where Z, X, Y, R⁸ is H and R¹¹ is as defined in formula I.
Step 1k: Reaction of the compound of formula I obtained in Step 1j with an acid to obtain a pharmaceutically acceptable salt of the compound of formula I of Step 1j.
Step 1m: Reaction of the compound of formula 7 with the compound of formula R³-halide; wherein R³ is —X—Y wherein X and Y are as defined in formula I, in presence of a base selected from anhydrous sodium carbonate, potassium carbonate, triethylamine or pyridine to afford the compound of formula I.
Step 1n: Reaction of the compound of formula I obtained in Step 1m with an acid to obtain a pharmaceutically acceptable salt of the compound of formula I of Step 1m.

The acid used in steps (1 h), (1k) and (1n) is selected from acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid or p-toluenesulfonic acid.

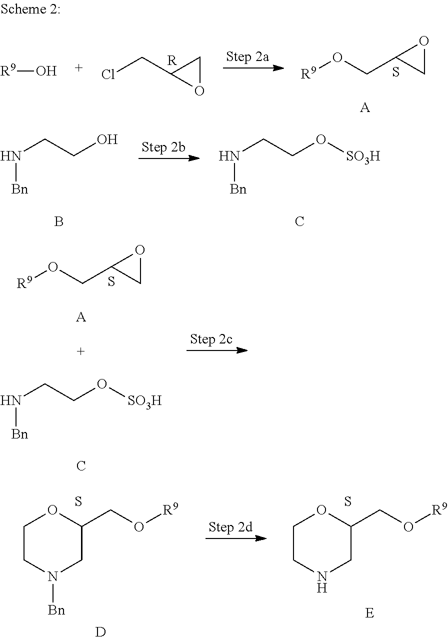

Scheme 2 describes the detailed process for the preparation of the compound of formula E used in Step 1d of Scheme 1 above, the steps comprising:

### Step 2a:

Reacting the compound of formula R⁹—OH wherein R⁹ is as defined in formula 1 (which is commercially available or may be prepared by methods well known in the art) with (R)-2-(chloromethyl)oxirane in presence of a base such as aqueous NaOH or aqueous KOH and a phase transfer catalyst such as tetrabutyl ammonium hydrogen sulphate at a temperature range of 80-120° C. for 1-4 h to afford the compound of formula A;

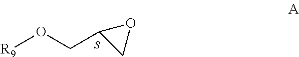

wherein R⁹ is as defined in formula I.

### Step 2b:

Reacting the compound of formula B (commercially available) with chlorosulfonic acid in a solvent selected from chloroform, carbon tetrachloride or dichloromethane, at 0-10° C. during addition of the acid over a period of 15-30 min, followed by at room temperature for 10-16 h to afford the compound of formula C;

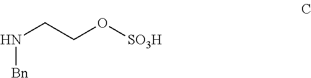 [SMILES: O=S(=O)(O)OCCNCc1ccccc1]

### Step 2c:

Reacting the compound of formula A with the compound of formula C in presence of an aqueous base such as NaOH or KOH in a suitable solvent selected from toluene, dioxane or THF in presence of a phase transfer catalyst such as tetrabutylammoniun hydrogen sulfate at a temperature range of 30-50° C. for 10-16 h to afford the compound of formula D;

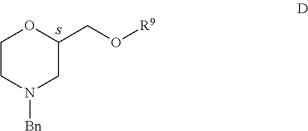

wherein R⁹ is as defined in formula I.

### Step 2d:

Carrying out debenzylation of the compound of formula D by refluxing the said compound of formula D with ammonium formate and 10% Pd/C in an atmosphere of carbon dioxide in a solvent selected from ethanol or methanol at 50-70° C. for 1-3 h to afford the compound of formula E:

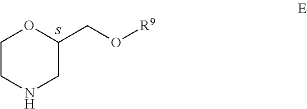

wherein R⁹ is as defined in formula I.

In an embodiment, Scheme 1A provides for the preparation of compounds 34 and 34a, which are representative examples of the Compound of formula I or formula IIA, wherein R¹ is chloro, R² is H, R³ is

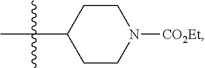 [SMILES: *C1CCN(C(=O)OCC)CC1]

and R⁹ is phenyl.

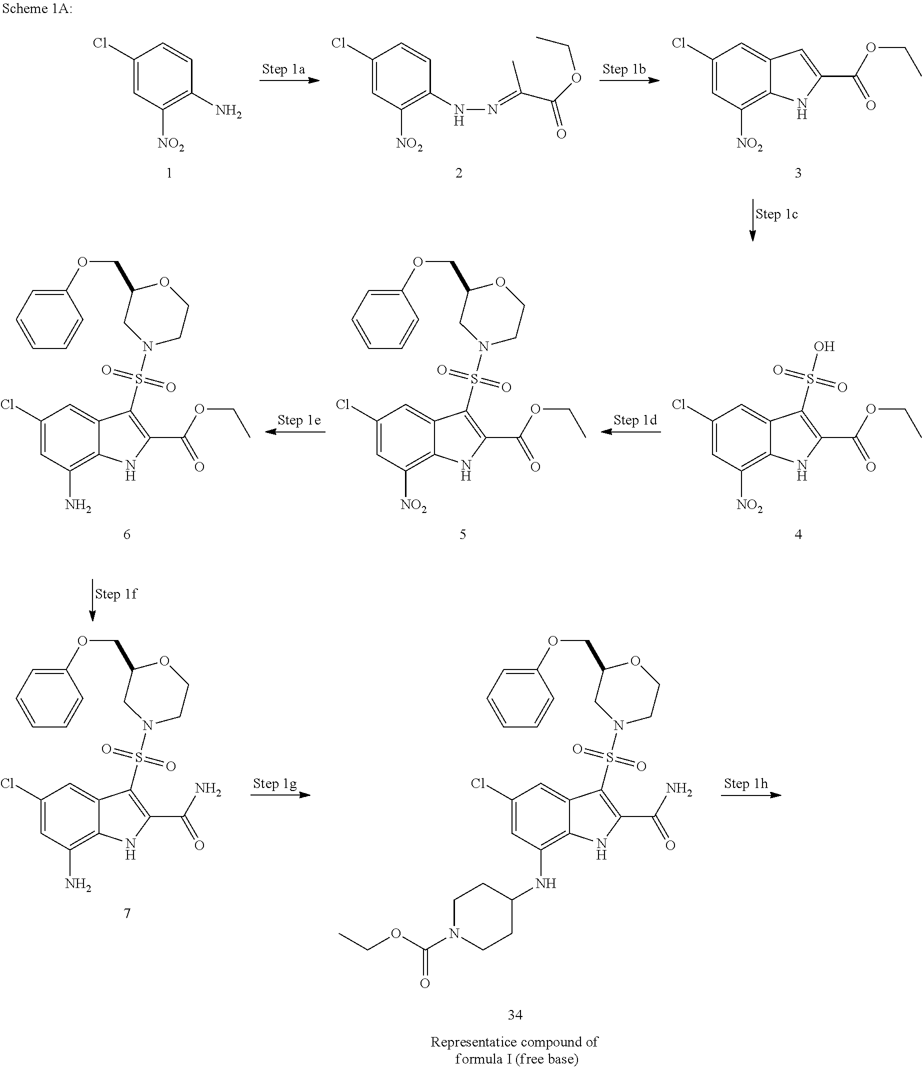

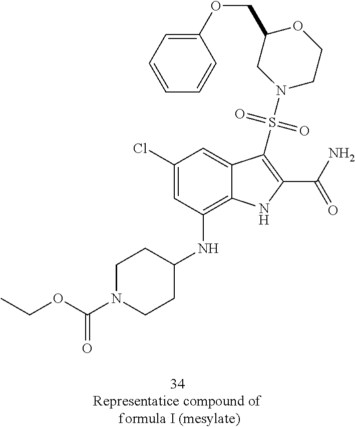 [SMILES: CCOC(=O)N1CCC(Nc2cc(Cl)cc3c(S(=O)(=O)N4CCO[C@H](COc5ccccc5)C4)c(C(N)=O)[nH]c23)CC1]

Scheme 1A describes the detailed process for the preparation of compounds 34 and 34a as the representative examples of the Compound of formula I, the steps comprising:

Step 1a: Diazotising the compound 1;

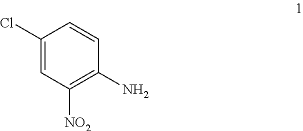 [SMILES: Nc1ccc(Cl)cc1[N+](=O)[O-]]

by reaction with NaNO₂ and HCl at a temperature range of −10° C. to 5° C. followed by reaction with ethyl 2-methyl-3-oxobutanoate at a temperature range of −20° C. to −15° C., which reaction is completed over a dropwise addition of the diazotized mixture to the reagent, ethyl 2-methyl-3-oxobutanoate in an alkaline solution of a base selected from KOH or NaOH in a solvent such as methanol or ethanol to afford the compound 2;

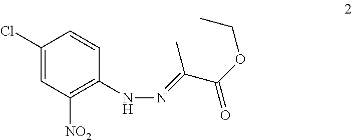 [SMILES: CCOC(=O)/C(C)=N/Nc1ccc(Cl)cc1[N+](=O)[O-]]

Step 1b: Cyclising the compound 2 by reaction with a Lewis acid such as ZnCl₂, AlCl₃, BF₃, P₂O₅ or polyphosphoric acid at a temperature range of 80-120° C. for 5-12 h to afford the compound 3;

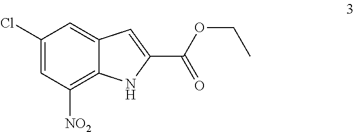 [SMILES: CCOC(=O)c1cc2cc(Cl)cc([N+](=O)[O-])c2[nH]1]

In an embodiment, cyclization is carried out using polyphosphoric acid as the Lewis acid at a temperature range of 100-110° C. for 8-9 h.

Step 1c: Sulphonating the compound 3 by reaction with sulphuric acid and acetic anhydride at a temperature range of 0-30° C. for 10-20 h to afford the compound 4;

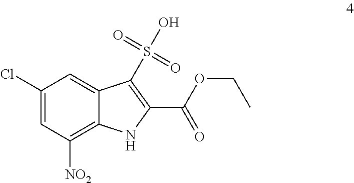 [SMILES: CCOC(=O)c1[nH]c2c([N+](=O)[O-])cc(Cl)cc2c1S(=O)(=O)O]

### Step 1d:

Reaction of the compound 4 with oxalyl chloride or thionyl chloride in presence of an organic base selected from triethylamine or pyridine in a solvent selected from DMF, methylene dichloride or a mixture thereof at a temperature range of 25-50° C. for 2-4 h to prepare the corresponding sulphonyl chloride of the compound 4, which is reacted with the reagent E;

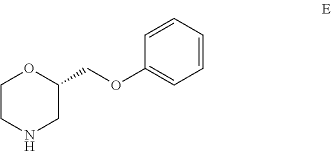 [SMILES: c1ccc(OC[C@@H]2CNCCO2)cc1]

in presence of an organic base selected from pyridine or triethylamine in a solvent selected from dichloromethane or chloroform at room temperature (25-30° C.) for 1-4 h to afford the compound 5;

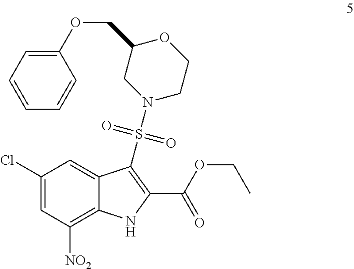 [SMILES: CCOC(=O)c1[nH]c2c([N+](=O)[O-])cc(Cl)cc2c1S(=O)(=O)N1CCO[C@H](COc2ccccc2)C1]

Step 1e: Reducing the compound 5 by reaction with a reducing agent selected from Fe and NH₄Cl, Zn and HCl or 5 nCl₂ for 2-8 h in a suitable solvent selected from methanol, ethanol, THF, water or a mixture thereof, to afford the compound 6.

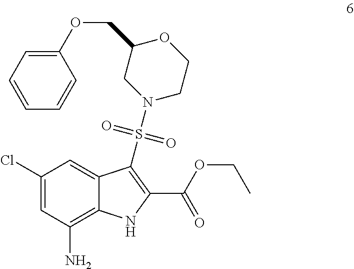 [SMILES: CCOC(=O)c1[nH]c2c(N)cc(Cl)cc2c1S(=O)(=O)N1CCO[C@H](COc2ccccc2)C1]

In an embodiment, reduction of the compound 5 is carried out using Fe and NH₄Cl as the reducing agent in a mixture of THF, water and ethanol as solvent at a temperature range of 70-80° C. for 2-4 h.

In an embodiment, the residual iron and iron oxides obtained along with compound 6 during reduction using Fe and NH₄Cl were removed by using EDTA and chloroform.

Step 1f: Reacting the compound 6 with isopropyl alcohol and ammonia at a temperature range of 80-120° C. in a sealed tube for 12-15 h or in a microwave for 10-15 min to afford the compound 7.

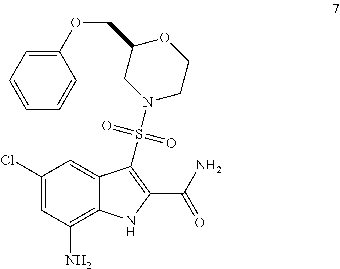 [SMILES: NC(=O)c1[nH]c2c(N)cc(Cl)cc2c1S(=O)(=O)N1CCO[C@H](COc2ccccc2)C1]

Step 1g: Reacting the compound of formula 7 with the reagent F;

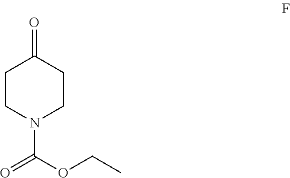

in the presence of trifluoroacetic acid in a base such as sodium triacetoxy borohydride in a solvent selected from dichloromethane or ethyl acetate and optionally with a Hunig's base at room temperature for 0.5-2 h to afford the compound 34, a representative compound of formula I as a free base wherein R¹ is chloro, R² is H, R³ is

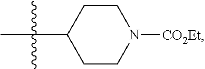 [SMILES: *C1CCN(C(=O)OCC)CC1]

and R⁹ is phenyl;

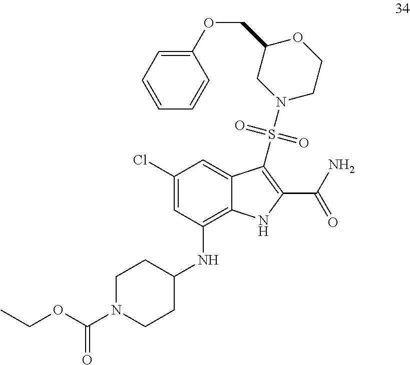 [SMILES: CCOC(=O)N1CCC(Nc2cc(Cl)cc3c(S(=O)(=O)N4CCO[C@H](COc5ccccc5)C4)c(C(N)=O)[nH]c23)CC1]

Step 1h: Reacting the compound 34 of Step 1g, in the form of a free base with methanesulphonic acid in THF as solvent at room temperature for about 30 min to 2 h to afford the corresponding methanesulfonate salt.

In an embodiment, Scheme 2A provides for the preparation of compound E used in Step 1d of Scheme 1A above.

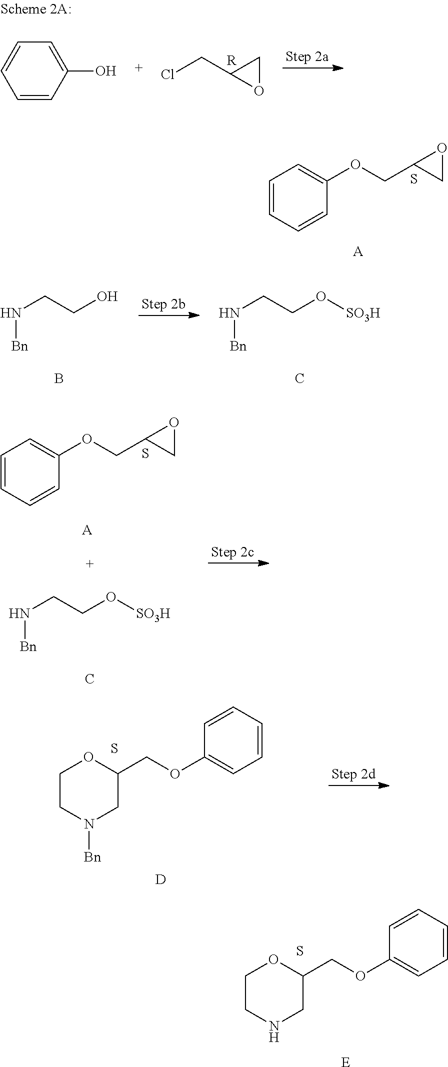

Scheme 2A provides the detailed process for the preparation of the Compound E used in Step 1d of Scheme 1A, the steps comprising:

### Step 2a:

Reacting commercially available phenol with (R)-2-(chloromethyl)oxirane in presence of a base selected from aqueous NaOH or aqueous KOH and a phase transfer catalyst such as tetrabutylammonium hydrogen sulphate at a temperature range of 80-120° C. for 1-4 h to afford the Compound A;

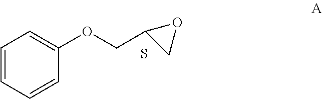

### Step 2b:

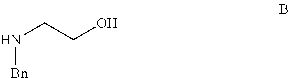 [SMILES: OCCNCc1ccccc1]

Reaction of the Compound B;

with chlorosulfonic acid in a solvent selected from chloroform, carbon tetrachloride, or dichloromethane, initially at 0-10° C. during addition of the acid, followed by at room temperature for 10-16 h to afford the Compound C;

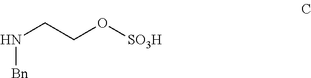 [SMILES: O=S(=O)(O)OCCNCc1ccccc1]

### Step 2c:

Reaction of the Compound A with the Compound C in presence of an aqueous base such as NaOH or aqueous KOH in a suitable solvent selected from toluene, dioxane or THF in presence of a phase transfer catalyst such as tetrabutylammoniun hydrogen sulfate at a temperature range of 30-50° C. for 10-16 h to afford the Compound D;

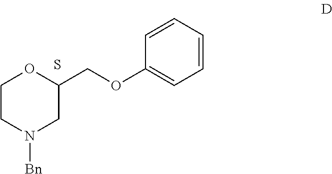

### Step 2d:

Debenzylation of the Compound D by refluxing the Compound D with ammonium formate and 10% Pd/C in an atmosphere of carbon dioxide in a solvent selected from ethanol or methanol at 50-70° C. for 1-3 h to afford the Compound E;

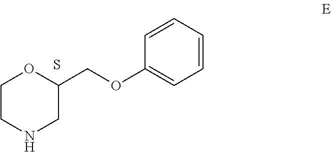

### EXAMPLES

### Example 1

### Ethyl 2-(2-(4-chloro-2-nitrophenyl)hydrazono)propanoate (Compound 2)

To an ice-cold solution of ethyl-2-methyl acetoacetate (965 g, 6.7 mol) in ethanol (4.0 L) was added 1.528 kg (50%) KOH at 0 to −10° C. This mixture was then diluted with 20.0 kg of ice. Simultaneously a cold diazonium salt solution was prepared from 2-nitro-4-chloro aniline (1 kg, 5.79 mol), 3.0 L of conc. HCl, 4.5 L of water and sodium nitrite (440 g, 6.37 mol) at 0 to −5° C. The diazonium salt mixture was then poured rapidly into the above ethanol solution of ethyl-2-methyl acetoacetate with constant stirring. The reaction was stirred for another 30 min. The solid was then filtered by suction filtration to yield crude compound 2, which was crystallised from ethanol to afford the title compound.

¹H NMR (300 MHz, DMSO-d₆): δ 10.87 (s, 1H), 8.19 (s, 1H), 8.01-7.99 (d, J=8.4 Hz, 1H), 7.57-7.54 (d, J=7.8 Hz, 1H), 4.37-4.35 (q, 2H), 2.24 (s, 3H), 1.40 (t, 3H); MS: m/z 284 (M−H)⁻.

### Example 2

### Ethyl 5-chloro-7-nitro-1H-indole-2-carboxylate (Compound 3)

Polyphosphoric acid (PPA) was heated at 110° C. and ethyl 2-(2-(4-chloro-2-nitrophenyl)hydrazono)propanoate (700 g, 2.45 mol) was added to the heated PPA mixture. This mixture was then stirred for 8-9 h. The reaction mass was basified using saturated sodium carbonate and the product was extracted in ethyl acetate (1 L×5). The organic layer was washed with saturated sodium carbonate (200 mL) followed by brine (200 mL), dried over anhydrous sodium sulphate and evaporated to afford the title compound.

¹H NMR (300 MHz, DMSO-d₆): δ 10.31 (s, 1H), 8.27-8.26 (d, J=1.5 Hz, 1H), 8.01-8.01 (d, J=1.2 Hz, 1H), 7.30-7.27 (s, 1H), 4.51-4.44 (q, 2H), 1.48-1.41 (t, 3H); MS: m/z 267 (M−H)⁻.

### Example 3

### 5-Chloro-2-(ethoxycarbonyl)-7-nitro-1H-indole-3-sulfonic acid (Compound 4)

To compound 3 of example 2 (350 g, 1.3 mol) was added acetic anhydride (622 mL, 6.529 mol) at room temperature. The reaction mixture was subsequently cooled to 0-10° C., and sulphuric acid (355 mL, 6.529 mol) was added drop wise. The reaction was stirred for 12-15 h at room temperature to ensure consumption of starting material. The solid was then filtered by suction filtration to yield the crude compound 3, which was crystallized using EtOAc (1-2 vol) to afford the title compound.

¹H NMR (300 MHz, DMSO-d₆): δ 12.28 (s, 1H),s 8.357-8.351 (d, J=1.8 Hz, 1H), 8.18-8.17 (d, J=1.8 Hz, 1H), 4.33-4.25 (q, 2H), 1.33-1.29 (t, 3H); MS: m/z 347 (M−H)⁻.

### Example 4

### (S)-Ethyl 5-chloro-7-nitro-3-(2-(phenoxymethyl)morpholinosulfonyl)-1H-indole-2-carboxylate (Compound 5)

To compound 4 of example 3 (175 g, 0.508 mol) was suspended in dichloromethane (700 mL) and catalytic amount of DMF was added. The reaction mixture was cooled to 10° C. and oxalyl chloride (130 mL, 1.508 mol) was added in a drop wise fashion. The reaction mixture was stirred for 12 h to afford the desired sulfonyl chloride. On completion of the reaction, the dichloromethane was distilled out completely. Fresh dichloromethane (500 mL), triethylamine (105 mL, 0.746 mol) and (S)-2-(phenoxymethyl)morpholine (102 g, 0.528 mol) was then added to the above solid and stirred for 4 h to ensure the coupling reaction. The dichloromethane was evaporated and the residue obtained was resuspended in water (200 mL) stirred and extracted in dichloromethane (500 mL×3). The organic layer was then washed with saturated bicarbonate (200 mL×2), brine (200 mL) and dried over anhydrous sodium sulfate (20 g). The organic layer was filtered and concentrated completely to afford the crude title compound.

¹H NMR (300 MHz, DMSO-d₆): δ 13.46 (s, 1H), 8.338-8.332 (d, J=1.8 Hz 1H), 8.26-8.25 (d, J=1.8 Hz, 1H), 7.29-7.24 (m, 2H), 6.95-6.88 (m, 3H), 4.41-4.34 (q, 2H), 3.98-3.93 (m, 3H), 3.81-3.77 (m, 2H), 3.67-3.58 (m, 2H), 2.60-2.49 (m, 2H), 1.32-1.28 (t, 3H); MS: m/z 524 (M+H)⁺.

### Example 5

### (S)-Ethyl 7-amino-5-chloro-3-(2-(phenoxymethyl)morpholinosulfonyl)-1H-indole-2-carboxylate (Compound 6)

Compound 5 of example 4 (150 g, 0.286 mol), iron powder (80 g, 1.435 mol), ammonium chloride (76.5 g, 1.435 mol) was mixed in ethanol (400 mL). The reaction mixture was heated up to 80-85° C. for 6-7 h. Ethanol was evaporated and the mixture was dissolved in chloroform (200 mL). To the chloroform layer, was added water in EDTA (200 g in 200 mL). The chloroform layer was separated. The water layer was further extracted with chloroform (200 mL×2). The combined organic layer was then washed with saturated sodium bicarbonate (200 mL×2), brine (200 mL) and subsequently dried over anhydrous sodium sulfate (20 g). The organic layer was then filtered and evaporated completely to afford the crude title compound.

¹H NMR (300 MHz, DMSO-d₆): δ 12.66 (s, 1H), 7.29-7.24 (m, 2H), 7.17 (s, 1H), 6.95-6.88 (m, 3H), 6.52 (s, 1H), 6.00 (bs, 2H), 4.41-4.34 (q, 2H), 3.99-3.90 (m, 3H), 3.81-3.78 (m, 2H), 3.61-3.52 (m, 2H), 2.59-2.50 (m, 2H), 1.34-1.22 (t, 3H); MS: m/z 494.1 (M+H)⁺.

### Example 6

### (S)-7-Amino-5-chloro-3-(2-(phenoxymethyl)morpholinosulfonyl)-1H-indole-2-carboxamide (7)

Compound 6 of example 5 (95 g, 0.192 mol) was dissolved in isopropyl alcohol (IPA) (900 mL) in a sealed tube and ammonia gas was passed through it for 15 min. The tube was sealed and heated to 110° C. for 12-15 h. The pressure was released carefully and isopropyl alcohol was evaporated. The solid was absorbed on silica (200-400 mesh) and purified using column chromatography (silica gel, 10% MeOH in chloroform) to afford the title compound.

¹H NMR (300 MHz, DMSO-d₆): δ 12.59 (s, 1H), 8.30-8.23 (d, J=21.0 Hz, 2H), 7.28-7.23 (m, 2H), 7.108-7.102 (d, J=1.8 Hz, 1H), 6.94-6.87 (m, 3H), 6.49-6.48 (d, J=1.8 Hz, 1H), 6.01 (bs, 2H), 4.03-3.94 (m, 2H), 3.90-3.79 (m, 2H), 3.68-3.46 (m, 3H), 2.50-2.31 (m, 2H); MS: m/z 465.1 (M+H)⁺.

### Example 7

### (S)-Ethyl 4-((2-carbamoyl-5-chloro-3-((2-(phenoxymethyl)morpholino) sulfonyl)-1H-indol-7-yl)amino)piperidine-1-carboxylate (Compound 34)

A mixture of compound 7 of example 6 (40 g, 0.0862 mol) and ethyl 4-oxopiperidine-1-carboxylate (29.51 g, 0.129 mol) were taken in dichloromethane (1.2 L) and the turbid solution was stirred for 20 h at room temperature. On completion of the reaction, TFA (33 mL) was added dropwise and stirred for 10 min. Following this, sodium triacetoxyborohydride (91 g, 0.431 mol) was added and the reaction mixture was stirred for another 1.5 h. The reaction mass was concentrated and the residue was dissolved in ethyl acetate (250 mL). The organic layer was washed with water (2×2.0 L) and brine (1.5 L). The organic phase was dried over anhydrous sodium sulphate and concentrated to yield a crude solid (56.0 g), which was purified using column chromatography (silica gel, 2% MeOH in CHCl₃) to afford the title compound.

¹H NMR (300 MHz, DMSO-d₆): δ 12.66 (s, 1H), 8.31-8.31 (d, J=12.6 Hz, 2H), 7.28-7.23 (t, J=8.1 Hz, 2H), 7.14-7.13 (d, J=1.2 Hz, 1H), 6.95-6.87 (m, 2H), 6.474-6.471 (d, J=0.9 Hz, 1H), 6.38-6.36 (d, J=7.2, 1H), 4.08-3.94 (m, 2H), 3.97-3.91 (m, 4H), 3.82-3.80 (m, 2H), 3.67-3.64 (d, J=10.5 Hz, 2H), 3.58-3.43 (m, 2H), 3.07 (m, 2H), 2.45-2.30 (m, 3H), 2.02-1.98 (d, J=9.9 Hz, 2H), 1.37-1.26 (m, 2H), 1.21-1.17 (t, J=6.9 Hz, 3H); MS: m/z 620.2 (M+H)⁺.

### Example 8

### Methanesulfonic acid salt of (S)-ethyl 4-((2-carbamoyl-5-chloro-3-((2-(phenoxymethyl) morpholino)sulfonyl)-1H-indol-7-yl)amino)piperidine-1-carboxylate (Compound 34a)

Compound 34 of example 7 (41 g, 0.0661 mol) was dissolved in THF (400 mL) and methane sulfonic acid (6.35 g, 0.0661 mol) was added and stirred at room temperature (RT) for 90 min. The content was concentrated to 200 mL and then 300 mL n-hexane was added and stirred till free powder was observed in the solution. The solid was filtered and washed with n-hexane (200 mL) and dried to afford the title compound.

¹H NMR (300 MHz, DMSO-d₆): δ 12.66 (s, 1H), 8.30-8.26 (d, J=13.2 Hz, 2H), 7.28-7.23 (t, J=7.5 Hz, 2H), 7.14 (s, 1H), 6.94-6.87 (m, 3H), 6.47 (s, 1H), 4.06-4.01 (m, 2H), 3.95-3.90 (m, 4H), 3.81 (m, 1H), 3.67-3.59 (m, 2H), 3.50-3.46 (m, 2H), 3.07 (m, 2H), 2.44 (s, 3H), 2.37-2.30 (m, 2H), 2.02-1.98 (d, J=10.5 Hz, 2H), 1.75 (m, 1H), 1.34-1.31 (m, 2H), 1.21-1.17 (t, J=7.2 Hz, 3H).

### Example 9

### (S)-2-Phenoxymethyloxirane (Compound A)

To a solution of NaOH (91.2 g, 2.28 mol) and phenol (143 g, 1.52 mol) in water (1.8 L), at room temperature was added tetrabutylammonium hydrogensulphate (1.5 g, 0.0044 mol). R-epichlorohydrin (662 g, 7.15 mol) was added slowly over a period of 10-15 min along with vigorous stirring. The mixture was stirred vigorously at 90-100° C. for 1 h. On completion of the reaction, it was extracted with 1:1 ethyl acetate: petroleum ether (1 L). The combined organic layer was concentrated below 40° C. to remove the solvent. The residue was distilled and the fraction from 115-125° C. at 2 mm (diaphragm pump) was collected (maintaining the oil bath temperature at 155-160° C.) to afford the title compound.

¹H NMR (300 MHz, CDCl₃): δ 7.28-7.34 (m, 2H), 6.93-7.03 (m, 3H), 4.255 (m, 1H), 4.00 (m, 1H), 3.390 (t, 1H), 2.95 (m, 1H), 2.785 (m, 1H); MS: m/z 151 (M+H).

### Example 10

### N-Benzyl Ethanolamine Hydrogen Sulphate (Compound C)

A solution of N-benzylethanolamine (328 g, 2.169 mol) in CCl₄ (2 L) was cooled to 0° C. Chlorosulphonic acid (256 g, 2.197 mol) was added dropwise to the solution while maintaining the reaction temperature between 0-5° C. After addition was complete, the mixture was stirred at room temperature for 16 h. On completion of the reaction, the solid was filtered, washed with 1:1 EtOH:CHCl₃ (650 mL) and dried at 50° C. under high vacuum (0.5 mm) for 1 h to afford the title compound.

¹H NMR (300 MHz, D₂O): δ 7.388 (s, 5H), 4.214 (m, 4H), 3.32 (t, 2H); MS: m/z 232 (M+H)⁺.

### Example 11

### (S)-1-Benzyl-2-phenoxymethylmorpholine (Compound D)

A solution of NaOH (572 g, 14.3 mol) in water (1 L) was cooled to 10-15° C. To this was added N-benzyl ethanolamine hydrogen sulphate (368 g, 1.591 mol) (C) while maintaining the temperature less than 20° C. The mixture was stirred at room temperature for 10 min A solution of (S)-2-(phenoxymethyl)oxirane (A) (216 g, 1.438 mol) in toluene was added over 10-15 min. The mixture was stirred at 45-50° C. for 16 h. On completion of the reaction, water (2 L) and EtOAc (2 L) was added to the reaction mixture. The organic layer was separated and washed with water and extracted with 10% aqueous HCl (2 L). The combined HCl washings were basified with NaOH to pH 9 and extracted with EtOAc (2.1 L). The EtOAc extract was washed with water (1 L), brine (1 L), dried over anhydrous Na₂SO₄ and concentrated completely to afford the title compound.

¹H NMR (300 MHz, CDCl₃): δ 7.33-7.23 (m, 7H), 6.96-6.93 (d, J=7.5 Hz, 1H), 6.90-6.88 (d, J=8.1 Hz, 2H), 4.05-3.90 (m, 4H), 3.77-3.66 (t, J=11.1 Hz, 1H), 3.55 (s, 2H), 3.49-2.86 (d, J=11.1 Hz, 1H), 2.70-2.66 (d, J=11.1 Hz, 1H), 2.274-2.187 (t, J=11.4 Hz, 1H), 2.131-2.063 (t, J=9.6 Hz, 1H); MS: m/z 284 (M+H)⁺.

### Example 12

### (S)-2-(Phenoxymethyl)morpholine (Compound E)

To a stirred solution of compound D (210 g, 0.741 mol) in methanol (2 L), under a bed of CO₂ (obtained by adding a small piece of dry ice to the mixture) was added 10% Pd/C. To the above reaction mixture was added ammonium formate (210 g, 3.3 mol) at ambient temperature and the above reaction mixture was refluxed for 1 h. On completion of the reaction, the Pd—C was filtered and washed with MeOH. The filtrate was concentrated and the residue obtained was dissolved in EtOAc (2 L). The organic layer was washed with water (1 L×2), dried over anhydrous Na₂SO₄ and concentrated at 60° C. for 1 h to afford the title compound.

¹H NMR (300 MHz, CDCl₃): δ 7.31-7.26 (m, 2H), 6.99-6.91 (m, 3H), 4.11-4.09 (m, 2H), d 4.047-3.990 (m, 2H), 3.977-3.656 (t, 1H), 3.091-2.740 (m, 4H); MS: m/z 194 (M+H)⁺.

### Example 13

### (S)-4-((2-Carbamoyl-5-chloro-3-((2-(phenoxymethyl)morpholino) sulfonyl)-1H-indol-7-yl)amino)-4-oxobutanoic acid (Compound 8)

Compound 7 of example 6 (0.075 g, 0.161 mol) was dissolved in toluene (5 mL) subsequent to which succinic anhydride (0.02 g, 0.200 mmol) was added and the reaction mixture was heated at 110° C. for 2 h. On completion of the reaction, toluene was evaporated, petroleum ether (20 mL) was added to the residue and the solid was filtered. The filtered solid was washed with 15 mL of petroleum ether to afford the title compound.

¹H NMR (300 MHz, DMSO-d₆): δ 12.84 (s, 1H), 12.25 (s, 1H), 10.18 (s, 1H), 8.34 (d, J=12.6 Hz, 2H), 8.13 (s, 1H), 7.65 (s, 1H), 7.28-6.87 (m, 5H), 3.94 (m, 3H), 3.81 (m, 1H), 3.70-3.49 (m, 3H), 2.71-2.60 (m, 4H), 2.44-2.27 (m, 2H).

### Example 14

### (S)-5-((2-Carbamoyl-5-chloro-3-(2-(phenoxymethyl)morpholino)sulfonyl)-1H-indol-7-yl)amino)-3,3-dimethyl-5-oxopentanoic acid (Compound 9)

The title compound was prepared analogous to the compound 8 of example 13 by reaction of compound 7 of example 6 (0.075 g, 0.161 mol) with 4,4-dimethyldihydro-2H-pyran-2,6(3H)-dione (24.1 mg, 0.169 mmol).

¹H NMR (300 MHz, DMSO-d₆): δ 12.85 (s, 1H), 12.04 (s, 1H), 10.01 (s, 1H), 8.37 (d, J=16.3 Hz, 2H), 8.20 (s, 1H), 7.66 (s, 1H), 7.25-6.87 (m, 3H), 3.94 (m, 3H), 3.81 (m, 2H), 3.69-3.49 (m, 4H), 2.38 (s, 4H), 1.14 (s, 6H).

### Example 15

### (S)-4-((2-Carbamoyl-5-chloro-3-(2-(phenoxymethyl)morpholino)sulfonyl)-1H-indol-7-yl)amino)-2,2-dimethyl-4-oxobutanoic acid (Compound 10)

The title compound was prepared analogous to the compound 8 of example 13 by reaction of compound 7 of example 6 (0.075 g, 0.161 mol) with 3,3-dimethyldihydrofuran-2,5-dione (21.6 mg, 0.169 mmol).

¹H NMR (300 MHz, DMSO-d₆): δ 13.06 (s, 1H), 12.09 (s, 1H), 8.31-8.25 (d, J=19.2 Hz, 2H), 7.94 (s, 1H), 7.44 (s, 1H), 7.26-6.91 (m, 5H), 3.97 (m, 2H), 3.91 (m, 1H), 3.83 (m, 1H), 3.74-3.52 (m, 4H), 2.78 (s, 2H), 2.44 (m, 2H), 1.14 (s, 6H).

### Example 16

### (S)-5-((2-Carbamoyl-5-chloro-3-(2-(phenoxymethyl)morpholino)sulfonyl)-1H-indol-7-yl)amino)-5-oxopentanoic acid (Compound 11)

The title compound was prepared analogous to the compound 8 of example 13 by reaction of compound 7 of example 6 (0.075 g, 0.161 mmol) with glutaric anhydride (19.34 mg, 0.169 mmol).

¹H NMR (300 MHz, DMSO-d₆): δ 12.90 (s, 1H), 12.09 (s, 1H), 10.07 (s, 1H), 8.36-8.33 (d, J=16.3 Hz, 2H), 8.15 (s, 1H), 7.66 (s, 1H), 7.28-6.87 (m, 5H), 3.95-3.90 (m, 3H), 3.81 (m, 1H), 3.70-3.49 (m, 3H), 2.40-2.32 (m, 2H), 1.89-1.65 (m, 6H).

### Example 17

### 2-((2-Carbamoyl-5-chloro-3-(((S)-2-(phenoxymethyl)morpholino)sulfonyl)-1H-indol-7-yl)carbamoyl)cyclopropanecarboxylic acid (Compound 12)

The title compound was prepared analogous to the compound 8 of example 13 by reaction of compound 7 of example 6 (0.075 g, 0.161 mmol) with 3-oxabicyclo[3.1.0]hexane-2,4-dione (19.0 mg, 0.169 mmol).

¹H NMR (300 MHz, DMSO-d₆): δ 12.86 (s, 1H), 12.28 (s, 1H), 10.37 (s, 1H), 8.38-8.34 (d, J=17.6 Hz, 2H), 8.11 (s, 1H), 7.66 (s, 1H), 7.28-6.87 (m, 5H), 3.95-3.90 (m, 3H), 3.83-3.81 (m, 1H), 3.70-3.49 (m, 4H), 2.30 (m, 1H), 2.16-2.08 (m, 2H), 1.51-1.45 (m, 1H), 1.30-1.26 (m, 1H).

### Example 18

### (S)-5-Chloro-7-(5-morpholino-5-oxopentanamido)-3-(2-(phenoxymethyl)morpholino sulfonyl)-1H-indole-2-carboxamide (Compound 13)

Compound 11 of example 16 (0.075 g, 0.129 mmol) was dissolved in DMF (0.5 mL), to which O-(benzotriazol-1-yl)-N,N,N′,N′-tetramethyluronium tetrafluoroborate (TBTU) (0.062 g, 0.194 mmol) was added and stirred at room temperature for 5 min. To this reaction mixture, morpholine (12.3 mg, 10.53 μL, 0.141 mmol) was added and stirred for about 16 h. On completion of the reaction, ice was added to the reaction mixture and the desired product was extracted using ethyl acetate. The organic layer was washed with water, dried over anhydrous sodium sulfate and evaporated to afford the title compound.

¹H NMR (300 MHz, DMSO-d₆): δ 12.53 (s, 1H), 10.06 (s, 1H), 8.33 (s, 2H), 8.15 (s, 1H), 7.65 (s, 1H), 7.26 (s, 2H), 6.89 (s, 3H), 3.95 (m, 3H), 3.82-3.79 (m, 1H), 3.70-3.67 (m, 1H), 3.54 (m, 7H), 3.44 (m, 5H), 2.40 (m, 6H).

### Example 19

### (S)-5-chloro-7-(2-cyanoacetamido)-3-((2-phenoxymethyl)morpholino)sulfonyl)-1H-indole-2-carboxamide (Compound 14)

Compound 7 of example 6 (50 mg, 0.101 mmol) was dissolved in DMF, to which O-(benzotriazol-1-yl)-N,N,N′,N′-tetramethyluronium tetrafluoroborate (TBTU) (29 mg, 0.111 mol) and Hunig's base (0.2 mL, 0.152 mmol) was added and stirred at room temperature for min. To this reaction mixture, 2-cyanoacetic acid (9.5 mg, 0.111 mol) was added and stirred for about 16 h. On completion of the reaction, ice was added to the reaction mixture and the desired product was extracted using ethyl acetate. The organic layer was washed with water, dried over anhydrous sodium sulfate and evaporated to afford the title compound.

Yield: 25 mg (44%); ¹H NMR (300 MHz, DMSO-d₆): δ 12.78 (s, 1H), 10.46 (s, 1H), 8.36-8.32 (d, J=11.7 Hz, 2H), 7.88 (s, 1H), 7.73 (s, 1H), 7.26 (m, 2H), 6.90-6.87 (m, 3H), 4.05-3.95 (m, 2H), 3.82-3.50 (m, 3H), 3.10 (m, 2H), 2.38-1.99 (m, 3H), 1.40-1.33 (m, 1H).

### Example 20

### (S)-Ethyl 5-(2-carbamoyl-5-chloro-3-(2-(phenoxymethyl)morpholinosulfonyl)-1H-indol-7-ylamino)-5-oxopentanoate (Compound 15)

To a solution of compound 11 of example 16 (0.075 g, 0.129 mmol) in ethanol (5 mL), concentrated sulfuric acid (catalytic, 0.5 mL) was added drop wise at 0° C. The reaction mixture was refluxed at 75° C. for 3 h. On completion of the reaction, a small portion of ice was added to the reaction mixture and extracted with EtOAc. The organic layer was washed with NaHCO₃ solution and brine solution to yield a crude residue, which was purified using column chromatography (silica gel, 10% MeOH in chloroform) to afford the title compound.

### Example 21

### (S)-3-(2-Carbamoyl-5-chloro-3-(2-(phenoxymethyl)morpholinosulfonyl)-1H-indol-7-ylamino)propanoic acid (Compound 16)

The titled compound was obtained in a two step procedure. The ethyl ester intermediate ((S)-ethyl 3-(2-carbamoyl-5-chloro-3-(2-(phenoxymethyl)morpholinosulfonyl)-1H-indol-7-yl amino)propanoate) was obtained by condensation of compound 7 of example 6 (0.075 g, 0.161 mol) with ethyl bromopropionate (0.033 g, 0.185 mol) in the presence of potassium carbonate under refluxing conditions. The ethyl ester intermediate ((S)-ethyl 3-(2-carbamoyl-5-chloro-3-(2-(phenoxymethyl)morpholinosulfonyl)-1H-indol-7-ylamino)propanoate) (0.080 g, 0.141 mol) was dissolved in ethanol (3 mL), and subjected to hydrolysis with 1M NaOH (8.5 mg) for 4 h to afford the desired compound. Upon completion, ethanol was evaporated. The aqueous layer was filtered through celite and subsequently acidified. The acidified layer was then filtered and purified using column chromatography (silica gel, 5% MeOH in chloroform) to afford the title compound.

¹H NMR (300 MHz, DMSO-d₆): δ 12.69 (s, 1H), 12.30 (s, 1H), 8.29-8.24 (d, J=19.5 Hz, 2H), 7.28-7.23 (m, 2H), 7.16 (s, 1H), 6.94-6.87 (m, 3H), 6.53 (m, 1H), 6.36 (s, 1H), 3.98-3.90 (m, 4H), 3.81 (m, 1H), 3.67 (m, 1H), 3.41 (m, 2H), 2.72 (m, 1H), 2.63-2.58 (m, 2H), 2.18 (m, 2H).

### Example 22

### (S)-7-((3-Amino-3-oxopropyl)amino)-5-chloro-3-((2-(phenoxymethyl)morpholino) sulfonyl)-1H-indole-2-carboxamide (Compound 17)

The titled compound was obtained in a two step procedure. The first step was to obtain the same ethyl ester intermediate ((S)-ethyl 3-(2-carbamoyl-5-chloro-3-(2-(phenoxymethyl)morpholinosulfonyl)-1H-indol-7-ylamino)propanoate)) as described in example 21. This ester intermediate was reacted with saturated isopropanolic ammonia in sealed tube at 110° C. for about 16 h to afford the titled compound. On completion of the reaction IPA/ammonia was evaporated and the title compound was obtained after purification using column chromatography (silica gel, 0-5% MeOH in CHCl₃).

¹H NMR (300 MHz, DMSO-d₆): δ 12.74 (s, 1H), 8.28-8.22 (d, J=13.6 Hz, 2H), 7.68 (s, 1H), 7.39-7.14 (m, 3H), 6.90-6.88 (m, 2H), 6.53 (s, 1H), 6.36 (s, 1H), 3.95-3.90 (m, 2H), 3.81 (m, 1H), 3.67-3.46 (m, 3H), 2.33 (m, 2H), 1.99-1.87 (m, 2H), 1.64-1.51 (m, 2H), 1.33-1.23 (m, 3H).

### Example 23

### (S)-Ethyl 4-((2-carbamoyl-5-chloro-3-(2-(phenoxymethyl)morpholino)sulfonyl)-1H-indol-7-yl)amino)butanoate (Compound 18)

Compound 7 of example 6 (100 mg, 0.45 mmol) was dissolved in DMF to which cesium carbonate (84.16 mg, 0.258 mmol) was added. The reaction mixture was cooled to 0° C. and 3-bromopropanenitrile (50 mg, 0.258 mmol) was added drop wise. The reaction mixture was stirred for 16 h at room temperature (25-30° C.). On completion of the reaction, DMF was evaporated completely. The solid residue was dissolved in EtOAc and residual solid was filtered off. The crude material was distilled to yield the crude title compound which was purified using column chromatography (silica gel, 2% MeOH in chloroform).

Yield: 29 mg (23%); ¹H NMR (300 MHz, DMSO-d₆): δ 12.62 (s, 1H), 8.30-8.26 (d, J=13.5 Hz, 2H), 7.26-7.14 (m, 3H), 6.90-6.87 (m, 3H), 6.44-6.35 (m, 2H), 4.08-4.05 (m, 2H), 3.5 (m, 3H), 3.80 (m, 1H), 3.67-3.46 (m, 3H), 3.20 (m, 2H), 2.37-2.33 (m, 2H), 1.89 (m, 2H), 1.23 (m, 2H), 1.20-1.15 (m, 3H).

### Example 24

### (S)-5-Chloro-7-((2-cyanoethyl)amino)-3-(2-(phenoxymethyl)morpholino)sulfonyl)-1H-indole-2-carboxamide (Compound 19)

Compound 7 of example 6 (0.075 g, 0.161 mmol) was dissolved in DMF to which potassium carbonate (0.055 g, 0.403 mmol) and potassium iodide (catalytic) was added. The reaction mixture was cooled to 0° C. and 3-bromopropanenitrile (0.039 g, 0.242 mmol) was added drop wise. The reaction mixture was stirred at 100° C. for 3 days. On completion of the reaction, DMF was evaporated completely and the solid residue was dissolved in dichloromethane. The residual solid was filtered off. The crude material was distilled to afford the title compound, which was purified using column chromatography (silica gel, 2% MeOH in chloroform).

¹H NMR (300 MHz, DMSO-d₆): δ 12.66 (s, 1H), 8.30-8.25 (d, 2H), 7.26-7.18 (m, 3H), 6.90-6.85 (m, 3H), 6.79-6.74 (m, 1H), 6.48 (s, 1H), 3.99-3.88 (m, 3H), 3.81-3.78 (m, 1H), 3.66-3.44 (m, 5H), 2.84-2.79 (m, 2H), 2.40-2.25 (m, 2H).

### Example 25

### (S)-5-Chloro-3-(2-(phenoxymethyl)morpholino)sulfonyl)-7-((tetrahydro-2H-pyran-4-yl)amino)-1H-indole-2-carboxamide (Compound 20)

Compound 7 of example 6 (0.075 g, 0.161 mol), dihydro-2H-pyran-4(3H)-one (0.024 g, 0.242 mmol) and Hunig's base (0.104 g, 0.808 mmol) were dissolved in dichloromethane and the reaction mixture was stirred for 2 h. Sodium triacetoxy borohydride (0.171 g, 0.805 mmol) was added to the reaction mixture and stirring was continued for 2 days. On completion of the reaction, the solvent was evaporated and the crude compound obtained was purified using column chromatography (silica gel, 2% MeOH in chloroform).

¹H NMR (300 MHz, DMSO-d₆): δ 12.67 (s, 1H), 8.30-8.25 (d, J=29.0 Hz, 2H), 7.26-7.23 (m, 2H, 7.13 (s, 1H), 6.90-6.87 (m, 3H), 6.46 (s, 1H), 6.39-6.37 (d, J=6.3 Hz, 1H), 3.95-3.82 (m, 5H), 3.67-3.46 (m, 4H), 2.41 (m, 2H), 2.34-2.30 (m, 1H), 2.00-1.91 (m, 2H), 1.46-1.42 (m, 2H), 1.23 (m, 2H).

### Example 26

### (S)-5-chloro-7-(cyclohexylamino)-3-((2-(phenoxymethyl)morpholino)sulfonyl)-1H-indole-2-carboxamide (Compound 21)

Compound 7 of example 6 (0.075 g, 0.161 mol), cyclohexanone (23.7 mg, 0.242 mmol) and Hunig's base (104.3 mg, 0.807 mmol) were dissolved in dichloromethane and the reaction mixture was stirred for 2 h. Sodium triacetoxy borohydride (171.1 mg, 0.807 mmol) was added to the reaction mixture and stirring was continued for 2 days. On completion of the reaction, the solvent was evaporated and the crude compound obtained was purified using column chromatography (silica gel, 2% MeOH in chloroform).

Yield: 9.6 mg (11%); ¹H NMR (300 MHz, DMSO-d₆): δ 12.69 (s, 1H), 8.29-8.24 (d, J=17.8 Hz, 2H), 7.28-7.23 (m, 2H), 7.10 (m, 1H), 6.95-6.87 (m, 3H), 6.36-6.31 (m, 2H), 3.97-3.94 (m, 3H), 3.90 (m, 1H), 3.67-3.45 (m, 3H), 2.41 (m, 1H), 2.37-2.30 (m, 2H), 2.03-1.99 (m, 2H), 1.73 (m, 2H), 1.63 (m, 2H), 1.43-1.34 (m, 2H), 1.28 (m, 2H).

### Example 27

### (S)-5-chloro-7-((cyclohexylmethyl)amino)-3-(2-(phenoxymethyl)morpholino)sulfonyl)-1H-indole-2-carboxamide (Compound 22)

Compound 7 of example 6 (0.075 g, 0.161 mol), 2-cyclohexylacetaldehyde (27.1 mg, 0.242 mmol) and Hunig's base (104.3 mg, 0.807 mmol) were dissolved in dichloromethane and the reaction mixture was stirred for 2 h. Sodium triacetoxy borohydride (171.1 mg, 0.807 mmol) was added to the reaction mixture and stirring was continued for 2 days. On completion of the reaction, the solvent was evaporated and the crude compound obtained was purified using column chromatography (silica gel, 2% MeOH in chloroform).

Yield: 43 mg (48%); ¹H NMR (300 MHz, DMSO-d₆): δ 12.69 (s, 1H), 8.30-8.25 (d, J=19.3 Hz, 2H), 7.28-7.23 (m, 2H), 7.12 (s, 1H), 6.95-6.87 (m, 3H), 6.38 (m, 1H), 6.30 (m, 1H), 3.82-3.79 (m, 1H), 3.67-3.46 (m, 3H), 3.02-3.99 (m, 2H), 2.41-2.30 (m, 4H), 1.89-1.85 (m, 2H), 1.74-1.63 (m, 4H), 1.33-1.26 (m, 3H), 1.07-1.00 (m, 3H).

### Example 28

### (S)-Methyl 4-(((2-carbamoyl-5-chloro-3-(2-(phenoxymethyl)morpholino)sulfonyl)-1H-indol-7-yl)amino)methyl)benzoate (Compound 23)

The title compound was prepared analogous to the compound 20 of example 25 by reaction of the compound 7 of example 6 (0.075 g, 0.161 mmol) with ethyl 4-formylbenzoate (0.039 g, 0.242 mmol), the crude compound obtained was purified using column chromatography (silica gel, 2% MeOH in chloroform).

¹H NMR (300 MHz, DMSO-d₆): δ 12.70 (s, 1H), 8.29-8.24 (d, J=15.6 Hz, 2H), 7.98-7.96 (d, J=8.1 Hz, 2H), 7.58-7.55 (d, J=8.4 Hz, 2H), 7.28-7.22 (m, 3H), 7.16 (s, 1H), 7.06 (m, 1H), 6.94-6.87 (m, 3H), 6.31 (s, 1H), 4.55-4.54 (d, J=4.8 Hz, 2H), 3.97-3.95 (m, 2H), 3.84 (m, 2H), 3.59-3.46 (m, 3H), 2.41-2.34 (m, 1H), 1.33-1.23 (m, 3H).

### Example 29

### (S)-5-chloro-7-(cyclopentylamino)-3-((2-(phenoxymethyl) morpholino)sulfonyl)-1H-indole-2-carboxamide (Compound 24)

Compound 7 of example 6 (0.075 g, 0.161 mol), cyclopentanone (20.3 mg, 0.242 mmol) and Hunig's base (104.3 mg, 0.807 mmol) were dissolved in DCM and the reaction mixture was stirred for 2 h. Then sodium triacetoxy borohydride (171.1 mg, 0.807 mmol) was added and stirring was continued for 2 days. Upon completion of reaction, the solvent was evaporated and the title compound was obtained after subjecting to column chromatography [2% MeOH in chloroform].

Yield: 37 mg (42%); ¹H NMR (300 MHz, DMSO-d₆): δ 12.67 (s, 1H), 8.30-8.25 (d, J=15.3 Hz, 2H), 7.28-7.23 (m, 2H), 7.12 (s, 1H), 6.95-6.87 (m, 3H), 6.45-6.43 (d, J=3.9 Hz, 2H), 6.32 (s, 1H), 3.97-3.94 (m, 3H), 3.84 (m, 2H), 3.67-3.46 (m, 3H), 2.60 (s, 1H), 2.36-2.29 (m, 1H), 2.00-1.98 (m, 2H), 1.72-1.54 (m, 4H), 1.34-1.30 (m, 2H).

### Example 30

### (S)-7-(((1-aminocyclopentyl)methyl)amino)-5-chloro-3-((2(phenoxymethyl)morpholino) sulfonyl)-1H-indole-2-carboxamide (Compound 25)

The title compound was prepared analogous to the compound 20 of example 25 by reaction of the compound 7 of example 6 (0.075 g, 0.161 mmol) with t-butyl(1-formylcyclopentyl)carbamate (0.051 g, 0.242 mol), to obtain the N-Boc protected intermediate of the title compound, which was treated with TFA in dichloromethane (1:1, v/v) to afford the amine, which was purified using column chromatography (silica gel, 2% MeOH in chloroform).

¹H NMR (300 MHz, DMSO-d₆) δ 12.67 (s, 1H), 8.24-8.13 (d, J=15.3 Hz, 2H), 7.25 (m, 2H), 7.13 (s, 1H), 6.90-6.87 (m, 3H), 6.40 (s, 1H), 6.19 (bs, 1H), 3.94 (m, 3H), 3.79 (m, 3H), 3.65 (m, 3H), 3.18 (m, 3H), 2.32-2.28 (m, 3H), 1.76 (m, 2H), 1.62-1.57 (m, 4H).

### Example 31

### (S)-4-(((2-Carbamoyl-5-chloro-3-((2-(phenoxymethyl)morpholino)sulfonyl)-1H-indol-7-yl)amino)methyl)benzoic acid (Compound 26)

The title compound was prepared analogous to the compound 20 of example 25 by reaction of the compound 7 of example 6 (0.075 g, 0.161 mmol) with 4-formylbenzoic acid (0.036 g, 0.242 mmol) to obtain a crude material, which was purified by column chromatography (silica gel, 2% MeOH in chloroform).

¹H NMR (300 MHz, DMSO-d₆): δ 12.73 (s, 1H), 8.30-8.23 (d, J=19.5 Hz, 2H), 7.95-7.93 (d, J=6.9 Hz, 2H), 7.54 (m, 2H), 7.25-7.04 (m, 3H), 6.89 (m, 2H), 6.33 (bs, 1H), 4.25 (s, 2H), 3.95-3.80 (m, 5H), 3.64 (m, 4H), 1.33 (m, 3H).

### Example 32

### (S)-7-((1-(tert-Butylcarbamoyl)piperidin-4-yl)amino)-5-chloro-3-((2-(phenoxymethyl)morpholino)sulfonyl)-1H-indole-2-carboxamide (Compound 27)

The title compound was prepared analogous to the compound 20 of example 25 by reaction of the compound 7 of example 6 (0.075 g, 0.161 mmol) with N-(tert-butyl)-4-oxopiperidine-1-carboxamide (0.048 g, 0.242 mmol) to obtain a crude material which was purified by column chromatography (Reverse phase C-18, 50 to 30% water in acetonitrile).

¹H NMR (300 MHz, DMSO-d₆): δ 12.67 (s, 1H), 8.31-8.26 (d, J=12.9 Hz, 2H), 7.28-7.23 (m, 2H), 7.13-7.12 (s, 1H), 6.95-6.87 (m, 3H), 6.46 (s 1H), 6.36-6.34 (d, 1H, J=6.0 Hz), 5.81 (s, 1H), 4.01-3.85 (m, 6H), 3.67-3.59 (m, 2H), 3.52-3.46 (m, 2H), 2.92-2.84 (t, 2H), 2.44-2.30 (m, 2H), 1.95-1.92 (d, 2H), 1.31 (m, 2H), 1.26 (s, 9H).

### Example 33

### (S)-5-Chloro-7-((1-(cyclohexylcarbamoyl)piperidin-4-yl)amino)-3-((2-(phenoxymethyl)morpholino)sulfonyl)-1H-indole-2-carboxamide (Compound 28)

The title compound was prepared analogous to the compound 20 of example 25 by reaction of the compound 7 of example 6 (0.075 g, 0.161 mmol) with N-cyclohexyl-4-oxopiperidine-1-carboxamide (0.072 g, 0.323 mmol) to obtain a crude material which was purified by column chromatography (Reverse phase C-18, 50 to 30% water in acetonitrile).

¹H NMR (300 MHz DMSO-d₆): δ 12.63 (s, 1H), 8.31-8.25 (d, J=15.0 Hz, 2H), 7.28-7.23 (m, 2H), 7.13 (s, 1H), 6.95-6.87 (m, 3H), 6.46 (s, 1H), 6.35-6.33 (d, J=6.0 Hz, 1H), 6.19-6.16 (d, J=9.0 Hz, 1H), 3.95-3.89 (m, 6H), 3.67-3.39 (m, 5H), 2.95-2.87 (t, 2H), 2.41-2.34 (m, 2H), 1.95-1.92 (d, 2H), 1.76-1.72 (t, 4H), 130-1.14 (m, 8H).

### Example 34

### (S)-5-Chloro-7-((1-((cyclohexylmethyl)carbamoyl)piperidin-4-yl)amino)-3-((2-(phenoxymethyl)morpholino)sulfonyl)-1H-indole-2-carboxamide (Compound 29)

The title compound was prepared analogous to the compound 20 of example 25 by reaction of the compound 7 of example 6 (0.075 g, 0.161 mmol) with N-(cyclohexylmethyl)-4-oxopiperidine-1-carboxamide (0.076 g, 0.323 mmol) to obtain a crude material which was purified by column chromatography (Reverse phase C-18, 50 to 30% water in acetonitrile).

¹H NMR (300 MHz DMSO-d₆): δ 12.6 (s, 1H), 8.30-8.21 (d, J=27.0 Hz, 2H), 7.27-7.21 (t, J=9.0 Hz, 2H), 7.12 (s, 1H), 6.93-6.85 (m, 3H), 6.50-6.46 (m, 2H), 6.34-6.32 (d, 1H), 3.92-3.78 (m, 9H), 2.40-2.34 (m, 2H), 1.95-1.92 (d, J=9.0 Hz, 2H), 1.66-1.63 (m, 6H), 1.32-1.13 (m, 12H).

### Example 35

### (S)-5-chloro-7-((4-fluorobenzyl)amino)-3-(2-(phenoxymethyl)morpholino)sulfonyl)-1H-indole-2-carboxamide (Compound 30)

(S)-7-amino-5-chloro-3-(2-(phenoxymethyl)morpholinosulfonyl)-1H-indole-2-carboxamide (7) (75 mg, 0.161 mmol), 4-fluorobenzaldehyde (30 mg, 0.240 mmol) and Hunig's base (104.3 mg, 0.807 mmol) were dissolved in dichloromethane and the reaction mixture was stirred for 2 h. Sodium triacetoxy borohydride (171.1 mg, 0.807 mmol) was added and stirring was continued for 2 days. On completion of the reaction, the solvent was evaporated and the title compound was obtained after subjecting to column chromatography (silica gel, 2% MeOH in chloroform].

Yield: 52 mg (57%); ¹H NMR (300 MHz, DMSO-d₆): δ 12.69 (s, 1H), 8.29-8.24 (d, J=16.2 Hz, 2H), 7.41 (m, 2H), 7.25-7.17 (m, 6H), 6.92-6.87 (m, 4H), 6.37 (s, 1H), 4.41 (s, 2H), 3.95-3.81 (m, 2H), 3.68-3.46 (5H), 2.38-2.34 (m, 1H).

### Example 36

### (S)-5-Chloro-7-((1-isobutylpiperidin-4-yl)amino)-3-((2-(phenoxymethyl)morpholino) sulfonyl)-1H-indole-2-carboxamide (Compound 31)

The title compound was prepared analogous to the compound 20 of example 25 by reaction of the compound 7 of example 6 (0.075 g, 0.161 mmol) with 1-isobutylpiperidin-4-one (0.037 g, 0.242 mmol) to obtain a crude material which was purified by column chromatography (silica gel, 2% MeOH in chloroform).

¹H NMR (300 MHz, DMSO-d₆): δ 12.65 (s, 1H), 8.29-8.20 (d, J=16.3 Hz, 2H), 7.26-7.21 (m, 2H), 7.10-7.097 (d, J=1.5 Hz, 2H), 6.93-6.85 (m, 3H), 6.35-6.32 (m, 2H), 3.99-3.88 (m, 3H), 3.78 (m, 2H), 3.66-3.44 (m, 5H), 3.38 (m, 2H), 3.08-3.00 (m, 4H), 2.79-2.76 (m, 2H), 2.54 (m, 1H), 2.39 (m, 1H), 0.86-0.79 (m, 6H).

### Example 37

### 5-Chloro-3-(((S)-2-(phenoxymethyl)morpholino)sulfonyl)-7-(pyrrolidin-3-ylamino)-1H-indole-2-carboxamide (Compound 32)

The title compound was prepared analogous to the compound 20 of example 25 by reaction of the compound 7 of example 6 (0.075 g, 0.161 mmol) with pyrrolidin-3-one (0.020 g, 0.242 mmol) to obtain a crude material which was purified by column chromatography (silica gel, 2% MeOH in chloroform).

¹H NMR (300 MHz, DMSO-d₆): δ 12.60 (s, 1H), 8.85-8.81 (m, 2H), 8.30 (s, 1H), 8.21-8.18 (d, J=7.8 Hz, 2H), 7.27-7.22 (m, 3H), 6.97-6.85 (m, 3H), 6.56 (m, 1H), 6.43 (s, 1H), 4.27 (m, 1H), 3.94-3.78 (m, 4H), 3.72-3.44 (m, 3H), 3.16 (m, 1H), 2.35-2.08 (m, 4H), 2.01-1.97 (m, 2H).

### Example 38

### (S)-Ethyl 4-(2-carbamoyl-5-fluoro-3-(2-(phenoxymethyl)morpholinosulfonyl)-1H-indol-7-ylamino)piperidine-1-carboxylate (Compound 33)

(S)-7-amino-5-fluoro-3-(2-(phenoxymethyl)morpholinosulfonyl)-1H-indole-2-carboxamide (0.15, 0.334 mmol) prepared by a method analogous to compound 7 of example 6 wherein the starting material used is 2-nitro-4-fluoro aniline, ethyl 4-oxopiperidine-1-carboxylate (0.085 g, 0.501 mmol, 0.86 mL), Hunig base (191 mL) and catalytic amount of DMAP were dissolved in dichloromethane (10 mL) and stirred at room temperature for 6 h. Subsequently sodium triacetoxyborohydride (0.105 g, 1.672 mmol) was added and stirred at room temperature for an additional 14 h. Dichloromethane was evaporated and the residual solid was dissolved in ethyl acetate (25 mL). The oraganic layer was washed with water (25 mL×2), brine (25 mL×2), dried over anhydrous Na₂SO₄ (1 g) and purified using column chromatography (silica gel, 0.5 to 1.5% methanol in chloroform) to afford the title compound.

¹H NMR (300 MHz, DMSO-d₆): δ 12.60 (s, 1H), 8.32-8.23 (d, J=27.0 Hz, 2H), 7.28-7.23 (m, 2H), 6.95-6.84 (m, 3H), 6.84-6.80 (m, 1H), 6.45-6.34 (m, 2H), 4.09-4.00 (m, 2H), 3.96-3.93 (m, 3H), 3.90 (m, 2H), 3.82-3.79 (m, 1H), 3.68-3.59 (m, 1H), 3.51 (m, 2H), 3.06 (m, 2H), 2.43-2.28 (m, 3H), 2.03-1.99 (m, 2H), 1.23-1.14 (m, 5H).

### Example 39

### (S)-5-Chloro-3-((2-(phenoxymethyl)morpholino)sulfonyl)-7-(3-phenylthioureido)-1H-indole-2-carboxamide (Compound 35)

Compound 7 of example 6 (0.075 g, 0.161 mmol) and isothiocyanatobenzene (43.7 mg, 0.323 mmol) were added together in dry THF and stirred for 12 h. The solid was filtered and washed with n-hexane to afford the title compound.

¹H NMR (300 MHz, DMSO-d₆): δ 12.95 (s, 1H), 10.18 (s, 1H), 9.63 (s, 1H), 8.23-8.19 (d, J=12.0 Hz, 2H), 7.72 (s, 1H), 7.57-7.54 (m, 2H), 7.48 (s, 1H), 7.39-7.34 (m, 2H), 7.29-7.24 (m, 2H), 7.19-7.14 (m, 1H), 6.95-6.89 (m, 3H), 3.98-3.97 (m, 2H), 3.86-3.81 (m, 2H), 3.74-3.70 (m, 2H), 3.63-3.56 (m, 2H), 2.27 (m, 1H).

### Example 40

### (S)-5-Chloro-3-((2-(phenoxymethyl)morpholino)sulfonyl)-7-(piperidin-4-ylamino)-1H-indole-2-carboxamide (Compound 36)

The title compound was prepared analogous to the compound 20 of example 25 by reaction of the compound 7 of example 6 (0.075 g, 0.161 mmol) with t-butyl 4-oxopiperidine-1-carboxylate (0.048 g, 0.242 mol), to obtain the N-Boc protected intermediate of the title compound, which was treated with TFA in dichloromethane (1:1, v/v) at room temperature for 4 h to afford the crude title compound, which was purified using column chromatography (silica gel, 0-5% MeOH in chloroform).

¹H NMR (300 MHz, DMSO-d₆): δ 12.61 (s, 1H), 8.50 (bs, 2H), 8.30-8.20 (m, 2H), 7.26-7.17 (m, 2H), 6.99-6.87 (m, 3H), 6.52-6.43 (m, 2H), 3.95-3.90 (m, 3H), 3.79 (m, 1H), 3.68-3.50 (m, 4H), 3.08 (m, 2H), 2.40-2.33 (m, 2H), 2.17-2.14 (m, 2H), 1.63-1.59 (m, 2H), 1.33-1.23 (m, 2H).

Other compounds of the invention can be synthesized using similar procedures as outlined above.

It should be noted that, as used in this specification and the appended claims, the singular forms “a”, “an”, and “the” include plural referents unless the content clearly dictates otherwise. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred aspects and techniques. However, it should be understood that many variations and modifications may be made while remaining within the spirit and scope of the invention.

1-7. (canceled)

8. A process for the preparation of a compound of formula I; 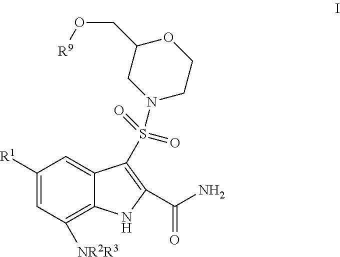
wherein:
R¹ is selected from the group consisting of:
H, halogen, NO₂, CN, (CR^{a}₂)ₙOR⁵, (CR^{a}₂)ₙN(R⁵)₂, C(O)R⁵, C(O)OR⁵, (CR^{a}₂)ₙR⁵, S(O)ₘR⁵, S(O)ₘN(R⁵)₂, SR⁵, OS(O)R⁵, N(R⁵)C(O)R⁵, N(R⁵)S(O)ₘR⁵, and (CR^{a}₂)ₙC(O)N(R⁵)₂;
R^{a} is independently selected from the group consisting of H and C₁-C₆ alkyl, said alkyl is optionally substituted with one to three substituents selected from R⁷;
R² is H or C₁-C₆ alkyl;
R³ is —C(Z)—X—C(O)—Y, —X—Y, —C(Z)—NR⁸R¹¹ or heterocyclyl, wherein said heterocyclyl is optionally substituted with one to three substituents selected from the group consisting of C₁-C₆ alkyl, NR⁸C(O)R¹⁰, C(O)NR⁸R¹⁰ and C(O)OR¹²;
R⁵ is independently selected from the group consisting of:
H, C₆-C₁₀aryl, 5-10 membered heterocyclyl, 5-10 membered heterocyclenyl, 5-10 membered heteroaryl, C₁-C₆alkyl, and C₃-C₈ cycloalkyl; wherein said aryl, heterocyclyl, heterocyclenyl, heteroaryl, alkyl and cycloalkyl are optionally substituted with one to three substituents selected from R⁷;
R⁷ is independently selected from the group consisting of: C₁-C₆ alkyl, halogen, C₁-C₆ alkoxy, C₁-C₆ haloalkyl, CN, NH₂, and NO₂;
R⁸ is independently H or C₁-C₆ alkyl;
R⁹ is selected from the group consisting of C₆-C₁₀aryl, 5-10 membered heterocyclyl, 5-10 membered heterocyclenyl and 5-10 membered heteroaryl, wherein said aryl, heterocyclyl, heterocyclenyl, and heteroaryl are optionally substituted with one to three substituents selected from R⁷;
R¹⁰ is independently selected from the group consisting of C₃-C₈cycloalkyl, C₁-C₆alkyl, and C₃-C₈cycloalkylC₁-C₃alkyl;
R¹¹ is selected from the group consisting of H, C₁-C₆ alkyl, C₆-C₁₀aryl, 5-10 membered heterocyclyl, 5-10 membered heterocyclenyl, and C₃-C₈cycloalkyl; wherein said alkyl, aryl, heterocyclyl, heterocyclenyl, and cycloalkyl are optionally substituted with one to three substituents selected from R⁷;
R¹² is H or C₁-C₆ alkyl;
X is C₂-C₆ alkylene or C₃-C₈cycloalkylene;
Y is selected from the group consisting of H, OR¹², CN, heterocyclyl, NR⁸R¹⁰, wherein said heterocyclyl is optionally substituted with one to three substituents selected from the group consisting of C(O)NR⁸R¹⁰, NR⁸C(O)R¹⁰, C₁-C₆ alkyl and C(O)OR¹²;
Z is NH, O or S;
in is independently 1 or 2;
n is independently 0, 1, 2, 3, 4, 5 or 6;
or a pharmaceutically acceptable salt thereof;
wherein said process comprises the steps of:
step 1a: diazotizing the compound of formula 1; 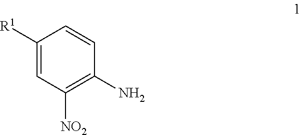
wherein R¹ is as defined in formula I, by reaction with sodium nitrite (NaNO₂) and hydrochloric acid (HCl) at a temperature range of −10 to 5° C., followed by a dropwise addition of the diazotized mixture to an alkaline solution of ethyl 2-methyl-3-oxobutanoate in a base selected from potassium hydroxide (KOH) or sodium hydroxide (NaOH) in a solvent such as methanol or ethanol at a temperature range of −20° C. to −15° C. to afford the compound of formula 2; 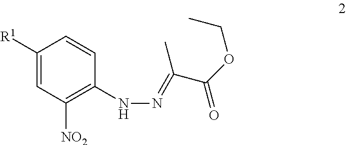
wherein R¹ is as defined in formula I;
step 1b: cyclising the compound of formula 2 by reaction with a Lewis acid selected from zinc chloride (ZnCl₂), aluminium chloride (AlCl₃), boron trifluoride (BF₃), phosphorus pentoxide (P₂O₅) or polyphosphoric acid at a temperature range of 80-120° C. for 5-12 h to obtain the compound of formula 3; 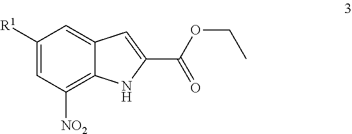
wherein R′ is as defined in formula I;
step 1c: sulphonating the compound of formula 3 by reaction with sulphuric acid and acetic anhydride at a temperature range of 0-30° C. for 10-20 h to obtain the compound of formula 4; 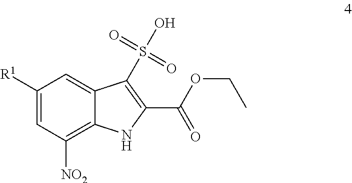
wherein R¹ is as defined in formula I;
step 1d: reacting the compound of formula 4 with oxalyl chloride or thionyl chloride in presence of an organic base selected from triethylamine or pyridine in a solvent selected from N,N-dimethylformamide (DMF), methylene dichloride or a mixture thereof at a temperature range of 25-50° C. for 1-6 h to prepare the corresponding sulphonyl chloride of the compound of formula 4, which is further reacted with the intermediate of formula E; 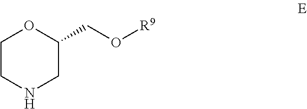
wherein R⁹ is as defined in formula I; at room temperature in the presence of an organic base selected from pyridine or triethylamine in a solvent selected from dichloromethane or chloroform at room temperature (25-30° C.) for 2-12 h to obtain the compound of formula 5; 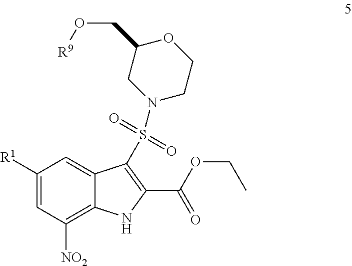
wherein R¹ and R⁹ are as defined in formula I;
step 1e: reducing the compound of formula 5 by reaction with a reducing agent selected from iron and ammonium chloride (Fe and NH₄Cl), zinc and hydrochloric acid (Zn and HCl) or stannous chloride (SnCl₂) for 2-8 h in a solvent selected from methanol, ethanol, tetrahydrofuran (THF), water or a mixture thereof, to obtain the compound of formula 6; 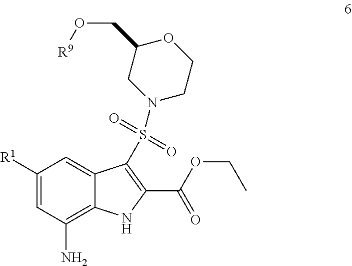
wherein R¹ and R⁹ are as defined in formula I;
step 1f: reacting the compound of formula 6 with isopropyl alcohol and ammonia at a temperature range of 80 to 120° C. in a sealed tube for 10-18 h or in a microwave for 10-15 min to obtain the compound of formula 7; 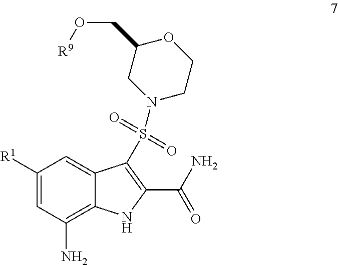
wherein R¹ and R⁹ are as defined in formula I;
step 1g: reacting the compound of formula 7 with the reagent of formula F; 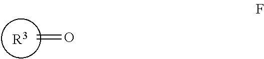
wherein R³ is an optionally substituted heterocyclyl or —X—Y, wherein X is (C₃-C₈)-cycloalkylene and Y is H, as defined in Formula I; in the presence of trifluoroacetic acid in sodium triacetoxy borohydride as a base and optionally, Hunig's base; in a solvent selected from dichloromethane or ethyl acetate at room temperature for 0.5-2 h to obtain the compound of formula I; wherein R¹ and R⁹ are as defined in formula I; R² is H and R³ is an optionally substituted heterocyclyl or —X—Y, wherein X is (C₃-C₈)-cycloalkylene and Y is H;
step 1h: reacting the compound of formula I obtained in step 1g with an acid to obtain a pharmaceutically acceptable salt of the compound of formula I of step 1g;
step 1j: reacting the compound of formula 7 as obtained in step 1f with the compound of formula: (R³)₂O, R³OH or R¹¹NC(Z) (wherein R³ is —C(Z)XC(O)Y or —C(Z)NR⁸R¹¹ where Z, X, Y, R⁸ is H and R¹¹ is as defined in formula I) in a solvent selected from toluene, dioxane or tetrahydrofuran (THF) at a temperature range of 70° C. to 100° C. for about 1-4 h to obtain the compound of formula I;
step 1k: reacting the compound of formula I obtained in step 1j with an acid to obtain a pharmaceutically acceptable salt of the compound of formula I of step 1j;
step 1m: reacting the compound of formula 7 as obtained in step 1f with the compound of formula: R³-halide; (R³ is —X—Y; wherein X and Y are as defined in formula I), in presence of a base selected from anhydrous sodium carbonate, potassium carbonate, triethylamine or pyridine to afford the compound of formula I; and
step 1n: reacting the compound of formula I obtained in step 1m with an acid to obtain a pharmaceutically acceptable salt of the compound of formula I of step l1.

9. The process according to claim 8, wherein the preparation of reagent E used in step 1d of claim 1 comprises the steps of:
step 2a: reacting the compound of formula R⁹—OH wherein R⁹ is as defined in formula 1 with (R)-2-(chloromethyl)oxirane in the presence of a base selected from aqueous sodium hydroxide (NaOH) or aqueous potassium hydroxide (KOH) and tetrabutyl ammonium hydrogen sulphate as the phase transfer catalyst, at a temperature range of 80-120° C. for 1-4 h to obtain the compound of formula A; 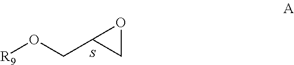
wherein R⁹ is as defined in formula I;
step 2b: reacting the compound of formula B, 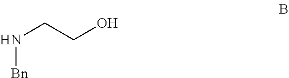 [SMILES: OCCNCc1ccccc1]
with chlorosulfonic acid in a solvent selected from chloroform, carbon tetrachloride or dichloromethane, at 0-10° C. during addition of the acid over a period of 15-30 min, followed by at room temperature for 10-16 h to afford the compound of formula C; 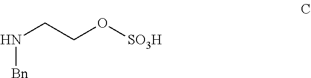 [SMILES: O=S(=O)(O)OCCNCc1ccccc1]
step 2c: reacting the compound of formula A obtained in step 2a with the compound of formula C obtained in step 2b in the presence of an aqueous base selected from sodium hydroxide (NaOH) or potassium hydroxide (KOH) in a solvent selected from toluene, dioxane or tetrahydrofuran (THF) in the presence of tetrabutylammoniun hydrogen sulfate as a phase transfer catalyst at a temperature range of 30-50° C. for 10-16 h to obtain the compound of formula D; 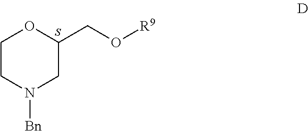
wherein R⁹ is as defined in formula I; and
step 2d: carrying out debenzylation of the compound of formula D by refluxing said compound of formula D with ammonium formate and 10% palladium on carbon (Pd/C) in an atmosphere of carbon dioxide in a solvent selected from ethanol or methanol at 50-70° C. for 1-3 h to obtain the compound of formula E; 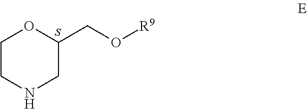
wherein R⁹ is as defined in formula I.

10. The process according to claim 8, wherein said process is provided for the preparation of the compound of formula (I) wherein IV is chloro, R² is H, R³ is 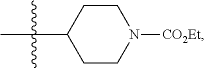 [SMILES: *C1CCN(C(=O)OCC)CC1]
and R⁹ is phenyl; comprising the steps of:
step 3a: diazotizing compound 1: 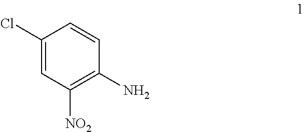 [SMILES: Nc1ccc(Cl)cc1[N+](=O)[O-]]
by reacting it with sodium nitrite (NaNO₂) and hydrochloric acid (HCl) at a temperature range of −10 to 5° C., followed by a dropwise addition of the diazotized mixture to an alkaline solution of ethyl 2-methyl-3-oxobutanoate in a base selected from potassium hydroxide (KOH) or sodium hydroxide (NaOH) in a solvent selected from methanol or ethanol at a temperature range of −20° C. to −15° C. to afford compound 2; 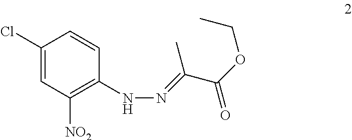 [SMILES: CCOC(=O)/C(C)=N/Nc1ccc(Cl)cc1[N+](=O)[O-]]
step 3b: cyclising the compound 2 by reacting it with a Lewis acid selected from zinc chloride (ZnCl₂), aluminium chloride (AlCl₃), boron trifluoride (BF₃), phosphorous pentoxide (P₂O₅) or polyphosphoric acid at a temperature range of 80-120° C. for 5-12 h to afford compound 3; 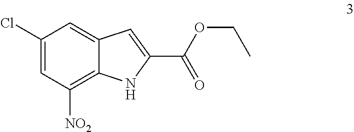 [SMILES: CCOC(=O)c1cc2cc(Cl)cc([N+](=O)[O-])c2[nH]1]
step 3c: sulphonating the compound 3 by reacting it with sulphuric acid and acetic anhydride at a temperature range of 0-30° C. for 10-20 h to afford compound 4; 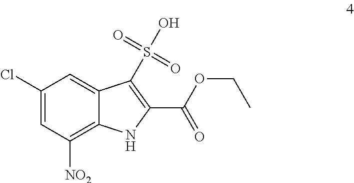 [SMILES: CCOC(=O)c1[nH]c2c([N+](=O)[O-])cc(Cl)cc2c1S(=O)(=O)O]
step 3d: reacting the compound 4 with oxalyl chloride or thionyl chloride in the presence of an organic base selected from triethylamine or pyridine in a solvent selected from N,N-dimethylformamide (DMF), methylene dichloride or a mixture thereof at a temperature range of 25-50° C. for 2-4 h to prepare the corresponding sulphonyl chloride of the compound 4, which is reacted with reagent E; 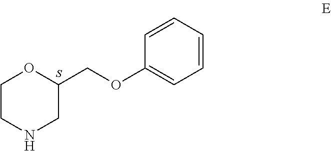
in the presence of an organic base selected from pyridine or triethylamine in a solvent selected from dichloromethane or chloroform at room temperature (25-30° C.) for 1-4 h to obtain compound 5; 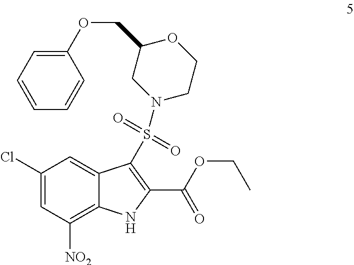 [SMILES: CCOC(=O)c1[nH]c2c([N+](=O)[O-])cc(Cl)cc2c1S(=O)(=O)N1CCO[C@H](COc2ccccc2)C1]
step 3e: reducing the compound 5 by reacting it with a reducing agent selected from iron and ammonium chloride (Fe and NH₄Cl), zinc and hydrochloric acid (Zn and HCl) or stannous chloride (SnCl₂) for 2-8 h in a solvent selected from methanol, ethanol, tetrahydrofuran (THF), water or a mixture thereof, to afford compound 6; 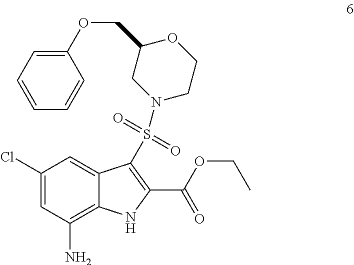 [SMILES: CCOC(=O)c1[nH]c2c(N)cc(Cl)cc2c1S(=O)(=O)N1CCO[C@H](COc2ccccc2)C1]
step 3f: reacting the compound 6 with isopropyl alcohol and ammonia at a temperature range of 80-120° C. in a sealed tube for 12-15 h or in a microwave for 10-15 min to afford compound 7; 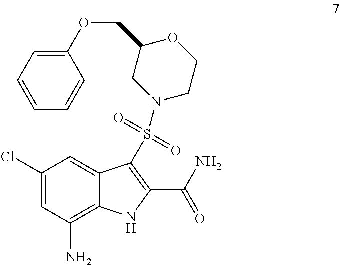 [SMILES: NC(=O)c1[nH]c2c(N)cc(Cl)cc2c1S(=O)(=O)N1CCO[C@H](COc2ccccc2)C1]
step 3g: reacting the compound 7 with reagent F; 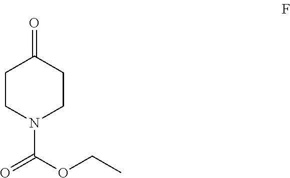
in the presence of trifluoroacetic acid in sodium triacetoxy borohydride as base in a solvent selected from dichloromethane or ethyl acetate and optionally with a Hunig's base at room temperature for 0.5-2 h to obtain the compound of formula (I) wherein R¹ is chloro, R² is H, R³ is 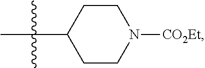 [SMILES: *C1CCN(C(=O)OCC)CC1]
and R⁹ is phenyl; and
step 3h: reacting the compound of formula (I) as obtained in step 3g with methanesulphonic acid in tetrahydrofuran (THF) as solvent at room temperature for about 30 min to 2 h to obtain the corresponding methanesulfonate salt.

11. The process according to claim 10, wherein the preparation of reagent E used in step 3d comprises the steps:
step 4a: reacting phenol with (R)-2-(chloromethyl)oxirane in presence of a base selected from aqueous sodium hydroxide (NaOH) or aqueous potassium hydroxide (KOH) and tetrabutylammonium hydrogen sulphate as the phase transfer catalyst, at a temperature range of 80-120° C. for 1-4 h to obtain Compound A; 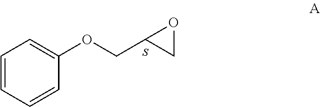
step 4b: reacting Compound B; 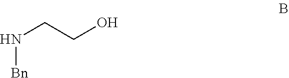 [SMILES: OCCNCc1ccccc1]
with chlorosulfonic acid in a solvent selected from chloroform, carbon tetrachloride, or dichloromethane, at 0-10° C. during addition of the acid over a period of 15-30 min, followed by at room temperature for 10-16 h to obtain Compound C; 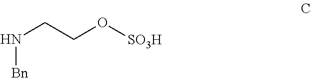 [SMILES: O=S(=O)(O)OCCNCc1ccccc1]
step 4c: reacting the Compound A obtained in step 4a with the Compound C obtained in step 4b in the presence of an aqueous base selected from sodium hydroxide (NaOH) or potassium hydroxide (KOH) in a solvent selected from toluene, dioxane or tetrahydrofuran (THF) in the presence of tetrabutylammoniun hydrogen sulfate as the phase transfer catalyst at a temperature range of 30-50° C. for 10-16 h to obtain Compound D; 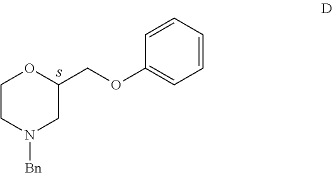
step 4d: carrying out debenzylation of the Compound D by refluxing the said Compound D with ammonium formate and 10% palladium on carbon (Pd/C) in an atmosphere of carbon dioxide in a solvent selected from ethanol or methanol at 50-70° C. for 1-3 h to afford reagent E;

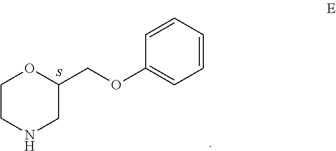

12. The process according to claim 8, wherein in step 1b, cyclization of the compound of formula 2 is carried out using polyphosphoric acid as the Lewis acid at a temperature range of 100-110° C. for 8-9 h.

13. The process according to claim 8, wherein in step 1e, reduction of the compound of formula 5 is carricd out using iron and ammonium chloride (Fe and NH₄Cl) as the reducing agent in a mixture of tetrahydrofuran (THF), water and ethanol as solvent at a temperature range of 70-80° C. for 2-4 h.

14. The process according to claim 8, wherein the acid used in step(s) (1h), (1k) and (1n) is selected from acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid or p-toluenesulfonic acid.
